(19) United States
(12) Patent Application Publication
(10) Pub. No.: US 2011/0009394 A1
(43) Pub. Date: Jan. 13, 2011

(54) TRICYCLIC NITROGEN COMPOUNDS AND THEIR USE AS ANTIBACTERIAL AGENTS

(75) Inventors: Pamela Brown, Hertfordshire (GB); Steven Dabbs, Hertfordshire (GB); Alan Joseph Hennessy, Hertfordshire (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited, Middlesex (GB)

(21) Appl. No.: 12/811,386

(22) PCT Filed: Jan. 7, 2009
(86) PCT No.: PCT/EP09/50091

§ 371 Date: Aug. 23, 2010

(30) Foreign Application Priority Data
Jan. 9, 2008 (GB) 0800367.5

(51) Int. Cl. A61K 31/542; C07D 471/16; C07D 513/04; C07D 497/04; C07D 491/052; C07D 491/056; A61K 31/519; A61P 31/04

(52) U.S. Cl. 514/224.2; 544/250; 544/48; 544/252; 514/250; 514/267

(57) ABSTRACT

Tricyclic nitrogen containing compounds and their use as antibacterials.

This invention relates to novel compounds, compositions containing them and their use as antibacterials including use in the treatment of tuberculosis.

WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006002047, WO2006014580, WO2006010040, WO2006017326, WO2006012396, WO2006017468, WO2006020561, WO2006081179, WO2006081264, WO2006081289, WO2006081178, WO2006081182, WO07016610, WO07081597, WO07071936, WO07115947, WO07118130, WO07122258, WO08006648, WO08003690, WO08009700, WO08116815, WO08125594, WO08128953, WO08128942, WO08128962, WO08128961, JP00212180, WO01/25227, WO02/40474, WO02/07572, WO04011454, WO2004024712, WO2004024713, WO2004035569, WO04060886, WO2004087647, WO2004089947, WO2005016916, WO2005097781, WO2006010831, WO2006021448, WO2006032466, WO2006038172, WO2006046552, WO06099884, WO06126171, WO06137485, WO06105289, WO06125974, WO06134378, WO07042325, WO07086016, WO07093963, WO07105154, WO07107965, WO07138974, WO08071962, WO08071964, WO08071981, WO08078305, WO08120003, WO08026172, WO08126024, WO08126034, US20080280879 disclose quinoline, naphthyridine, morpholine, cyclohexane, piperidine and piperazine derivatives having antibacterial activity. WO2004104000 discloses tricyclic condensed ring compounds capable of selectively acting on cannabinoid receptors.

This invention provides a compound of formula (I) or a pharmaceutically acceptable salt and/or N-oxide thereof:

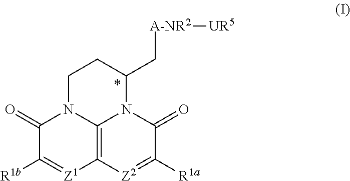

wherein:
Z¹ and Z² are selected from CH and N; one of R^{1a} and R^{1b} is selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted (C₁₋₆)alkyl; (C₁₋₆)alkoxy-substituted (C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl; and the other is hydrogen;
provided that R^{1a} and R^{1b} are H when Z² or Z¹ is N, respectively, and R^{1b} is H when Z² and Z¹ are both CH;
R² is hydrogen, or (C₁₋₄)alkyl, or together with R⁶ forms Y as defined below;
A is a group (i):

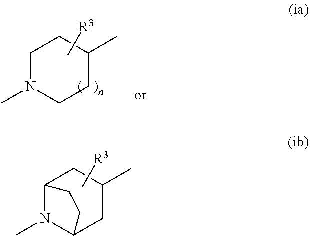

in which: R³ is as defined for R^{1a} and R^{1b} or is oxo and n is 1 or 2:
or A is a group (ii)

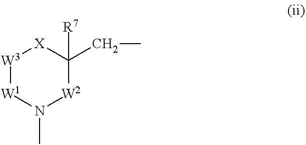

W¹, W² and W³ are CR⁴R⁸

or W² and W³ are CR⁴R⁸ and W¹ represents a bond between W³ and N.

X is O, CR⁴R⁸, or NR⁶;

one R⁴ is as defined for R^{1a} and R^{1b} and the remainder and R⁸ are hydrogen or one R⁴ and R⁸ are together oxo and the remainder are hydrogen;

R⁶ is hydrogen or (C₁₋₆)alkyl; or together with R² forms Y;

R⁷ is hydrogen; halogen; hydroxy optionally substituted with (C₁₋₆)alkyl; or (C₁₋₆)alkyl;

Y is CR⁴R⁸CH₂; CH₂CR⁴R⁸; (C═O); CR⁴R⁸; CR⁴R⁸(C═O); or (C═O)CR⁴R⁸;

or when X is CR⁴R⁸, R⁸ and R⁷ together represent a bond;

U is selected from CO, and CH₂ and
R⁵ is an optionally substituted bicyclic carbocyclic or heterocyclic ring system (B):

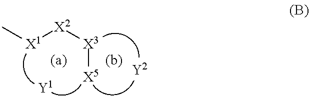

containing up to four heteroatoms in each ring in which

at least one of rings (a) and (b) is aromatic;

X¹ is C or N when part of an aromatic ring, or CR¹⁴ when part of a non-aromatic ring;

X² is N, NR¹³, O, S(O)ₓ, CO or CR¹⁴ when part of an aromatic or non-aromatic ring or may in addition be CR¹⁴R¹⁵ when part of a non aromatic ring;

X³ and X⁵ are independently N or C;

Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;

Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;

each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or

R¹⁴ and R¹⁵ may together represent oxo;

each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₄)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and

each x is independently 0, 1 or 2.

This invention also provides a method of treatment of bacterial infections including tuberculosis in mammals, particularly in man, which method comprises the administration to a mammal in need of such treatment an effective amount of a compound of formula (I), or a pharmaceutically acceptable salt and/or N-oxide thereof.

The invention also provides the use of a compound of formula (I), or a pharmaceutically acceptable salt and/or N-oxide thereof, in the manufacture of a medicament for use in the treatment of bacterial infections including tuberculosis in mammals.

The invention also provides a pharmaceutical composition comprising a compound of formula (I), or a pharmaceutically acceptable salt and/or N-oxide thereof, and a pharmaceutically acceptable carrier.

In a particular aspect the stereochemistry at the carbon atom marked * is S.

In particular aspects:

(i) Z¹ and Z² are both CH;

(ii) Z¹ is N and Z² is CH;

(iii) Z¹ is CH and Z² is N.

In a particular aspect R^{1a} and R^{1b} are independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen.

In particular embodiments R^{1a} and R^{1b} are hydrogen.

In a particular aspect R² is hydrogen.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen, hydroxy or fluoro.

In a particular aspect, when A is (ia), n is 1. In a further aspect R³ is in the 3- or 4-position. In a more particular aspect, A is (ia), n is 1 and R³ is in the 3-position, and more particularly is cis to the NR² group. In particular embodiments, A is a group (ia) in which n is 1 and R³ is hydrogen or hydroxy. More particularly where A is 3-hydroxy-piperidin-4-yl the configuration is (3R,4S) or (3S,4R). Alternatively and more particularly where A is piperidin-4-yl the configuration is (3R,4S).

In an alternative more particular aspect, when A is (ia), n is 1,R³ is in the 4-position and is methyl.

In a particular aspect, when A is (ii), X is CR⁴R⁸ and R⁸ is H and R⁴ is H or OH and more particularly OH is trans to R7. In a further aspect W¹ is a bond. In another aspect R⁷ is H. In an additional aspect W¹ is a bond, W² and W³ are both CH₂ and R⁷ is H. Where A is 4-hydroxypyrrolidin-3-ylmethyl, in a particular aspect the configuration is (3S,45). Where A is pyrrolidin-3-ylmethyl, in a particular aspect the configuration is 3S.

In a particular aspect, when A is (ii), X is O, R⁷ is H and W¹, W² and W³ are each CH₂.

In certain embodiments U is CH₂.

In certain embodiments R⁵ is an aromatic heterocyclic ring (B) having 8-11 ring atoms including 2-4 heteroatoms of which at least one is N or NR¹³ in which, in particular embodiments, Y² contains 2-3 heteroatoms, one of which is S and 1-2 are N, with one N bonded to X³.

In alternative embodiments the heterocyclic ring (B) has ring (a) aromatic selected from optionally substituted benzo, pyrido, pyridazino and pyrimidino and ring (b) non aromatic and Y² has 3-5 atoms, more particularly 4 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³. In a particular aspect the ring (a) contains aromatic nitrogen, and more particularly ring (a) is pyridine or pyrazine. Examples of rings (B) include optionally substituted:

### (a) and (b) Aromatic

1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, naphthalen-2-yl, 1,3-dioxo-isoindol-2yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-6-yl, pyrido[1,2-a]pyrimidin-4-one-3-yl (4-oxo-4H-pyrido[1,2-a]pyrimidin-3-yl), benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimidin-4-one-2-yl (4-oxo-4H-pyrido[1,2-a]pyrimidin-2-yl), quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, thiazolo[4,5-b]pyridin-5-yl, [1,2,3]thiadiazolo[5,4-b]pyridin-6-yl, 2H-isoquinolin-1-one-3-yl (1-oxo-1,2-dihydro-isoquinolin-3-yl), [1,2,3]thiadiazolo[5,4-b]pyridine-6-yl, 1,8-naphthyridin-2-yl, 2,1,3-benzooxadiazol-5-yl, 2(1H)-quinoxalinone-3-yl,

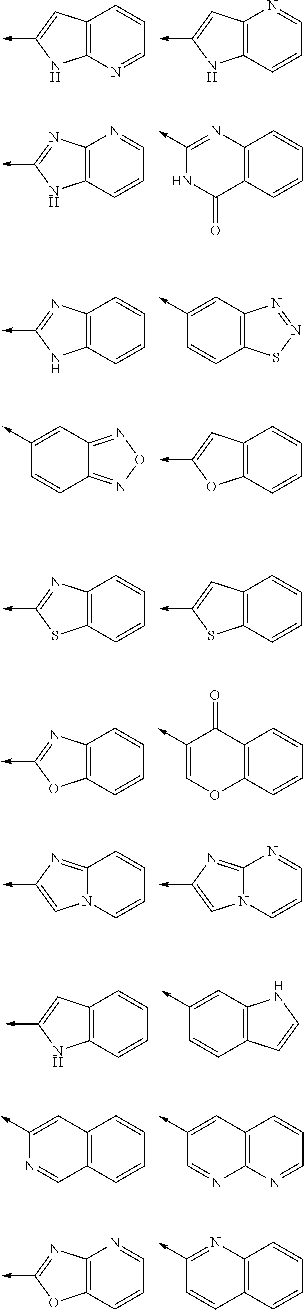

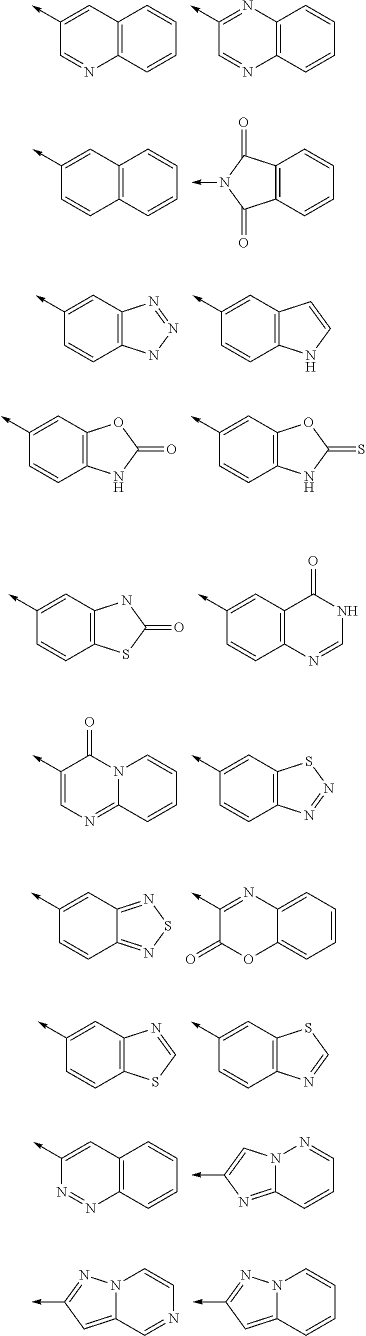

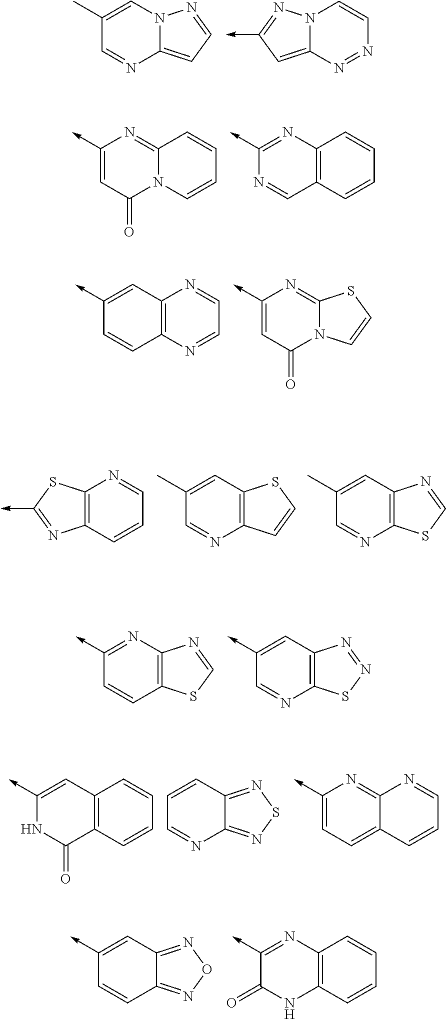

→ is the point of attachment

### (a) is Non Aromatic

(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl, (7S)-6,7-dihydro[1,4]dioxino[2,3-c]pyridazin-7-yl, indan-2-yl

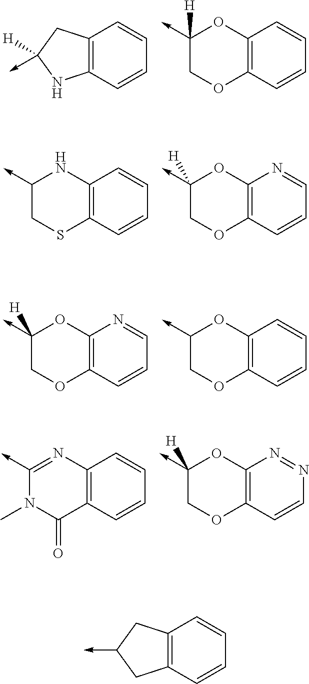

→ is the point of attachment

### (b) is Non Aromatic

1,1,3-trioxo-1,2,3,4-tetrahydro1 l⁶-benzo[1,4]thiazin-6-yl, benzo[1,3]dioxol-5-yl, 2,3-dihydro-benzo[1,4]dioxin-6-yl, 3-substituted-3H-benzooxazol-2-one-6-yl, 3-substituted-3H-benzooxazole-2-thione-6-yl, 3-substituted-3H-benzothiazol-2-one-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, 1H-pyrido[2,3-b][1,4]thiazin-2-one-7-yl (2-oxo-2,3-dihydro-1H-pyrido[2,3-b]thiazin-7-yl), 2,3-dihydro-1H-pyrido[2,3-b][1,4]thiazin-7-yl, 2-oxo-2,3-dihydro-1H-pyrido[3,4-b]thiazin-7-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 3,4-dihydro-2H-benzo[1,4]oxazin-6-yl, 3,4-dihydro-2H-benzo[1,4]thiazin-6-yl, 3-oxo-3,4-dihydro-2H-pyrido[3,2-b][1,4]oxazin-6-yl, 3,4-dihydro-2H-pyrido[3,2-b][1,4]thiazin-6-yl, 3-oxo-3,4-dihydro-2H-pyrido[3,2-b][1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl (1,2,3,4-tetrahydro-[1,8]naphthyridin-7-yl), 2-oxo-3,4-dihydro-1H-[1,8]naphthyridin-6-yl, 6-oxo-6,7-dihydro-5H-pyridazino[3,4-b][1,4]thiazin-3-yl (6-oxo-6,7-dihydro-5H-8-thia-1,2,5-triaza-naphthalen-3-yl), 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6,7-dihydro-[1,4]dioxino[2,3-d]pyrimidin-2-yl, [1,3]oxathiolo[5,4-c]pyridin-6-yl, 3,4-dihydro-2H-pyrano[2,3-c]pyridin-6-yl, 2,3-dihydro[1,4]oxathiino[2,3-c]pyridin-7-yl, 2,3-dihydrofuro[2,3-c]pyridin-5-yl, 2,3-dihydro-1-benzofuran-5-yl, 2,3-dihydro[1,4]oxathiino[2,3-b]pyridin-7-yl, 6,7-dihydro[1,4]oxathiino[3,2-c]pyridazin-3-yl, 6,7-dihydro[1,4]oxathiino[2,3-c]pyridazin-3-yl, 6,7-dihydro[1,4]dioxino[2,3-c]pyridazin-3-yl, 2,3-dihydro-1H-pyrido[3,4-b][1,4]oxazin-7-yl, 5-oxo-1,2,3,5-tetrahydroindolizin-7-yl, 6,7-dihydro-5H-pyrano[2,3-c]pyridazin-3-yl, 7-oxo-1,5,6,7-tetrahydro-1,8-naphthyridin-2-yl, 2,3-dihydro[1,4]oxathiino[3,2-c]pyridin-7-yl, 5,6-dihydrofuro[2,3-c]pyridazin-3-yl, 1-oxo-1,2,3,4-tetrahydro-7-isoquinolinyl, 7-oxo-1,5,6,7-tetrahydropyrido[2,3-d]pyrimidin-2-yl (2-substituted 5,6-dihydropyrido[2,3-c]pyrimidin-7(1H)-one), 6,7-dihydro-5H-pyrano[2,3-d]pyrimidin-2-yl, 2-methyl-1-oxo-1,2,3,4-tetrahydro-7-isoquinolinyl, 5,6-dihydro-4H-cyclopenta[b]thien-2-yl, 6,7-dihydro-5H-thieno[3,2-b]pyran-2-yl, 6,7,8-tetrahydroisoquinolin-3-yl, 6,7-dihydro-5H-cyclopenta[c]pyridin-3-yl, 1,3-dihydrofuro[3,4-c]pyridin-6-yl, 3,4-dihydro-2H-[1,4]oxathiepino[2,3-c]pyridin-8-yl, [1,3]oxathiolo[4,5-c]pyridin-6-yl, 2-substituted 1H-pyrimido[5,4-b][1,4]oxazin-7(6H)-one, 7-substituted 2H-chromen-2-one, 7-substituted 2H-pyrano[2,3-b]pyridin-2-one, 8-substituted 2H-pyrido[1,2-a]pyrimidin-2-one, 1H-pyrimido[5,4-b][1,4]thiazin-7(6H)-one-2-yl, 3,4-dihydro-2H-chromen-7-yl, 2,3-dihydro-1-benzofuran-6-yl, 3,4-dihydro-2H-chromen-6-yl, 2,3,4,5-tetrahydro-1,5-benzothiazepine-7-yl.

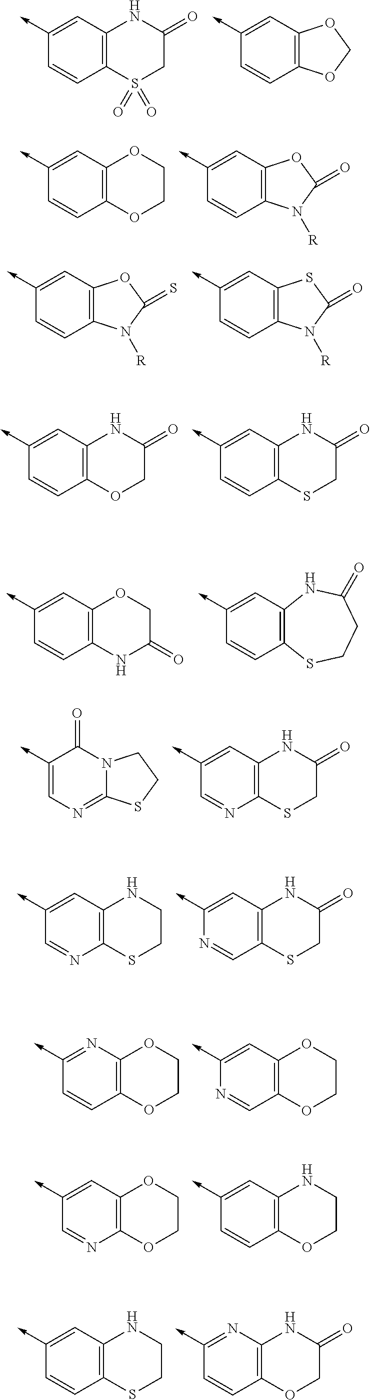

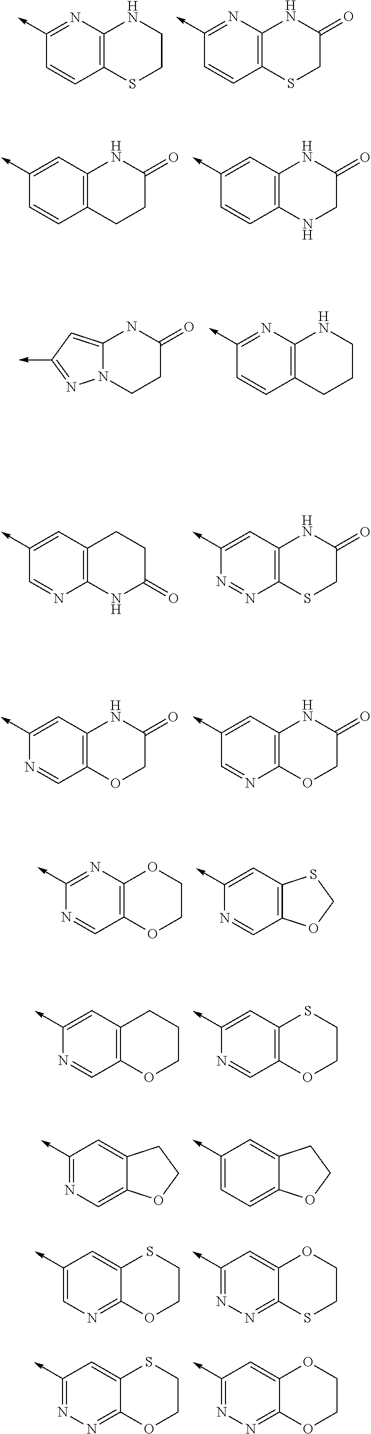

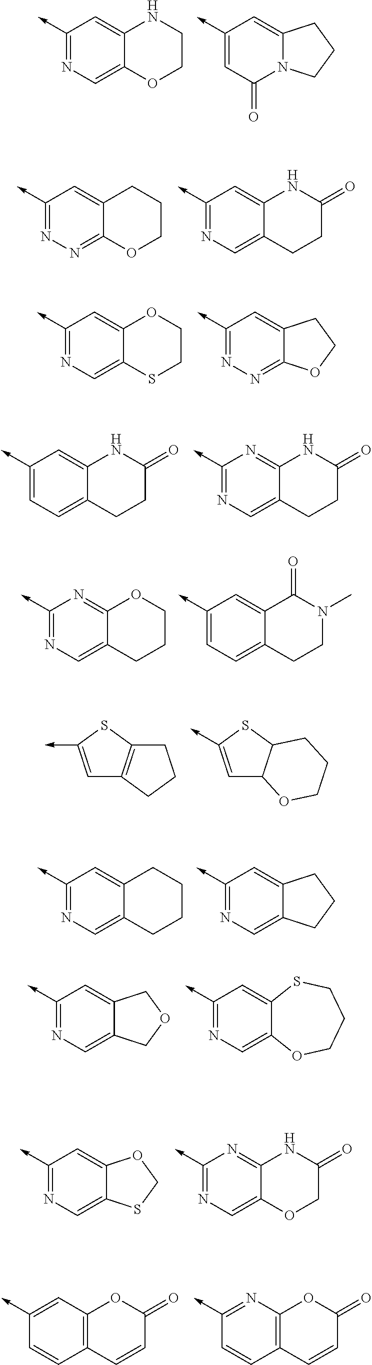

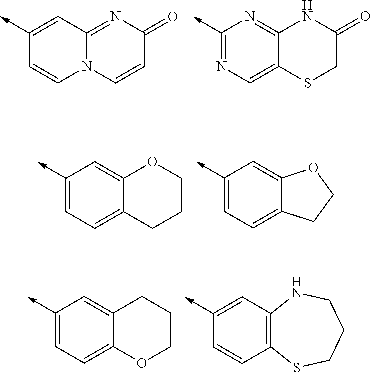

where R is an optional substituent
→ is the point of attachment

In some embodiments R¹³ is H if in ring (a) or in addition (C₁₋₄)alkyl such as methyl or isopropyl when in ring (b). More particularly, in ring (b) R¹³ is H when NR¹³ is bonded to X³ and (C₁₋₄)alkyl when NR¹³ is bonded to X⁵.

In further embodiments R¹⁴ and R¹⁵ are independently selected from hydrogen, halo, hydroxy, (C₁₋₄) alkyl, (C₁₋₄)alkoxy, nitro and cyano. More particularly R¹⁵ is hydrogen.

More particularly each R¹⁴ is selected from hydrogen, chloro, fluoro, hydroxy, methyl, methoxy, nitro and cyano. Still more particularly R¹⁴ is selected from hydrogen, fluorine or nitro.

Most particularly R¹⁴ and R¹⁵ are each H.

Particular groups R⁵ include:
[1,2,3]thiadiazolo[5,4-b]pyridin-6-yl
1H-pyrrolo[2,3-b]pyridin-2-yl
2,3-dihydro-[1,4]dioxino[2,3-b]pyridin-6-yl
2,3-dihydro-[1,4]dioxino[2,3-b]pyridin-7-yl
2,3-dihydro-[1,4]dioxino[2,3-c]pyridin-7-yl
2,3-dihydro-benzo[1,4]dioxin-6-yl
2-oxo-2,3-dihydro-1H-pyrido[2,3-b][1,4]oxazin-7-yl
2-oxo-2,3-dihydro-1H-pyrido[2,3-b][1,4]thiazin-7-yl
3,4-dihydro-2H-benzo[1,4]oxazin-6-yl
3-methyl-2-oxo-2,3-dihydro-benzooxazol-6-yl
3-oxo-3,4-dihydro-2H-benzo[1,4]oxazin-6-yl
3-oxo-3,4-dihydro-2H-pyrido[3,2-b][1,4]oxazin-6-yl
3-oxo-3,4-dihydro-2H-benzo[1,4]thiazin-6-yl (4H-benzo[1,4]thiazin-3-one-6-yl)
4-oxo-4H-pyrido[1,2-a]pyrimidin-2-yl
6-nitro-benzo[1,3]dioxol-5-yl
7-fluoro-3-oxo-3,4-dihydro-2H-benzo[1,4]oxazin-6-yl
8-hydroxy-1-oxo-1,2-dihydro-isoquinolin-3-yl
8-hydroxyquinolin-2-yl
benzo[1,2,3]thiadiazol-5-yl
benzo[1,2,5]thiadiazol-5-yl
benzothiazol-5-yl
thiazolo-[5,4-b]pyridin-6-yl
3-oxo-3,4-dihydro-2H-pyrido[3,2-b][1,4]thiazin-6-yl
7-chloro-3-oxo-3,4-dihydro-2H-pyrido[3,2-b][1,4]thiazin-6-yl
7-chloro-3-oxo-3,4-dihydro-2H-pyrido[3,2-b][1,4]oxazin-6-yl
7-fluoro-3-oxo-3,4-dihydro-2H-pyrido[3,2-b][1,4]thiazin-6-yl
2-oxo-2,3-dihydro-1H-pyrido[3,4-b][1,4]thiazin-7-yl
[1,3]oxathiolo[5,4-c]pyridin-6-yl
3,4-dihydro-2H-pyrano[2,3-c]pyridin-6-yl
5-carbonitro-2,3-dihydro-1,4-benzodioxin-7-yl
2,3-dihydro[1,4]oxathiino[2,3-c]pyridin-7-yl
6,7-dihydro[1,4]dioxino[2,3-c]pyridazin-3-yl
5,6,7,8-tetrahydroisoquinolin-3-yl
6,7-dihydro-5H-cyclopenta[c]pyridin-3-yl
1,3-dihydrofuro[3,4-c]pyridin-6-yl
6-fluoro-2,3-dihydro-1,4-benzodioxin-7-yl
3,4-dihydro-2H-[1,4]oxathiepino[2,3-c]pyridin-8-yl,
[1,3]oxathiolo[4,5-c]pyridine-6-yl
2,3-dihydro-1-benzofuran-5-yl
6,7-dihydro[1,4]oxathiino[2,3-c]pyridazin-3-yl
6,7-dihydro-5H-pyrano[2,3-c]pyridazin-3-yl
5,6-dihydrofuro[2,3-c]pyridazin-3-yl
2-substituted 1H-pyrimido[5,4-b][1,4]oxazin-7(6H)-one
2-substituted 4-chloro-1H-pyrimido[5,4-b][1,4]oxazin-7(6H)-one
2-substituted 5,6-dihydropyrido[2,3-d]pyrimidin-7(1H)-one
2-substituted 4-chloro-5,6-dihydropyrido[2,3-d]pyrimidin-7(1H)-one
2-substituted 4-methyl-5,6-dihydropyrido[2,3-d]pyrimidin-7(1H)-one
2-substituted 4-methyloxy-5,6-dihydropyrido[2,3-d]pyrimidin-7(1H)-one
7-substituted 2H-chromen-2-one
7-substituted 2H-pyrano[2,3-b]pyridin-2-one
4-chloro-6,7-dihydro-5H-pyrano[2,3-d]pyrimidin-2-yl
8-substituted 2H-pyrido[1,2-a]pyrimidin-2-one
6,7-dihydro-5H-pyrano[2,3-d]pyrimidin-2-yl)
5-chloro-1-benzothiophen-2-yl
6-chloro-1-benzothiophen-2-yl
1-benzothiophen-5-yl
1-methyl-1H-1,2,3-benzotriazol-6-yl
imidazo[2,1-b][1,3]thiazol-6-yl
4-methyl-3,4-dihydro-2H-1,4-benzoxazin-7-yl
1-methyl-1H-indol-2-yl
1H-pyrimido[5,4-b][1,4]thiazin-7(6H)-one-2-yl
[1,2,5]thiadiazolo[3,4-b]pyridine-6-yl
4-fluoro-1H-benzimidazol-2-yl
3,4-dihydro-2H-chromen-7-yl
2,3-dihydro-1-benzofuran-6-yl
3,4-dihydro-2H-chromen-6-yl
6-chloro-2,3-dihydro-1,4-benzodioxin-7-yl
7-chloro-3,4-dihydro-2H-pyrido[3,2-b][1,4]oxazine-6-yl
7-chloro-3,4-dihydro-2H-pyrido[3,2-b][1,4]thiazine-6-yl
3,4-dihydro-2H-pyrido[3,2-b][1,4]thiazin-6-yl
5-fluoro-2,3-dihydro-1,4-benzodioxin-7-yl
5-fluoro-2,3-dihydro-1,4-benzodioxin-6-yl
8-fluoro-2H-1,4-benzoxazin-3(4H)-one-6-yl
8-fluoro-3,4-dihydro-2H-1,4-benzoxazin-6-yl
7,8-difluoro-3,4-dihydro-2H-1,4-benzoxazin-6-yl
6,7-dihydro-5H-thieno[3,2-b]pyran-2-yl
5-methyl-2,3-dihydro-1,4-benzodioxin-7-yl
4-oxo-2,3,4,5-tetrahydro-1,5-benzothiazepin-7-yl
3,4-dihydro-2H-1,4-benzothiazine-6-yl
2,3,4,5-tetrahydro-1,5-benzothiazepine-7-yl
7-fluoro-3,4-dihydro-2H-1,4-benzoxazine-6-yl
6-fluoro-2,3-dihydro-1,4-benzodioxin-7-yl

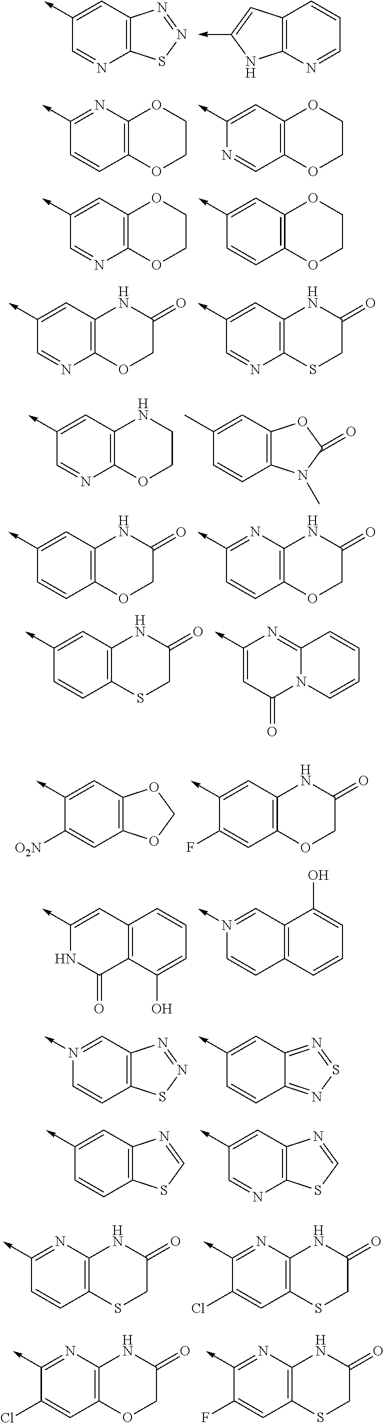

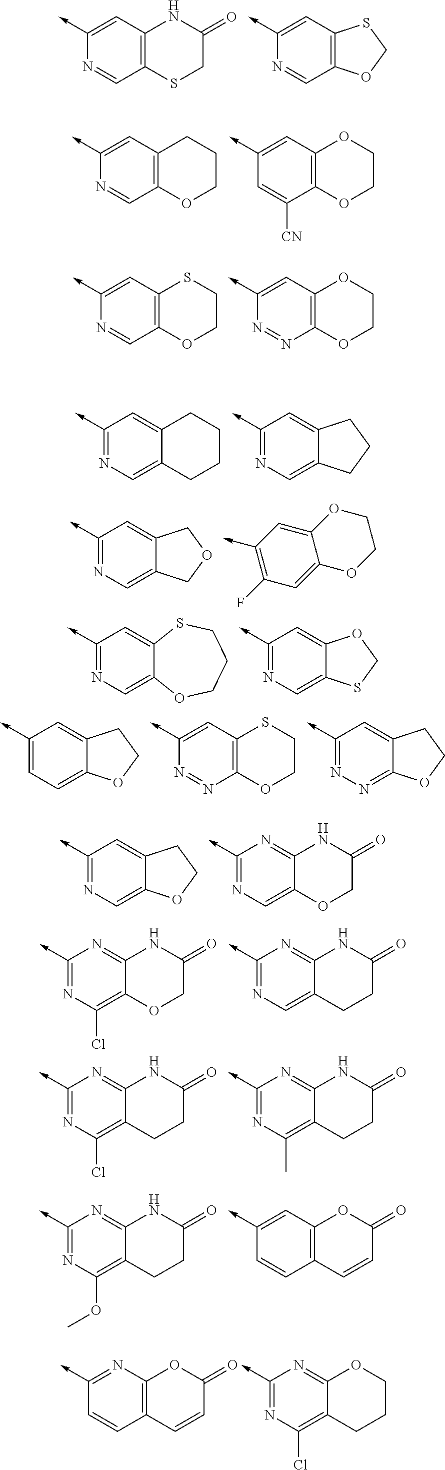

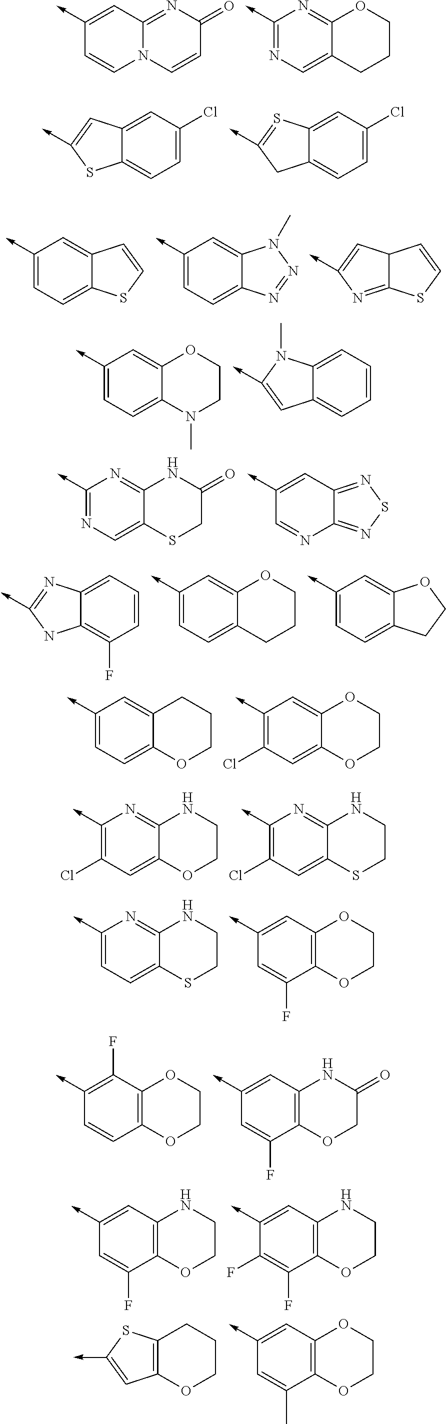

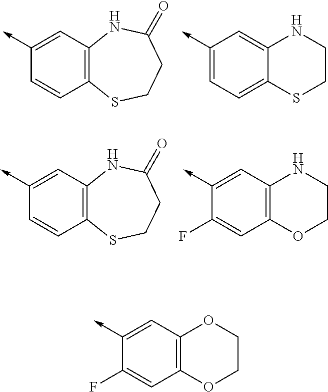

→ is the point of attachment
especially
3-oxo-3,4-dihydro-2H-pyrido[3,2-b][1,4]oxazin-6-yl
3-oxo-3,4-dihydro-2H-pyrido[3,2-b][1,4]thiazin-6-yl
2,3-dihydro-[1,4]dioxino[2,3-c]pyridin-7-yl
[1,3]oxathiolo[5,4-c]pyridin-6-yl
6-fluoro-2,3-dihydro-1,4-benzodioxin-7-yl
2,3-dihydro[1,4]oxathiino[2,3-c]pyridin-7-yl
3,4-dihydro-2H-pyrano[2,3-c]pyridin-6-yl

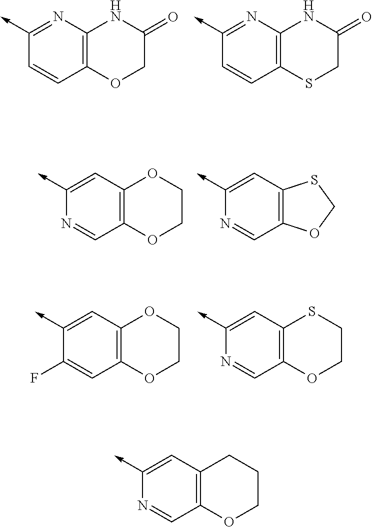

→ is the point of attachment

When used herein, the term “alkyl” includes groups having straight and branched chains, for instance, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of formula (I) or a pharmaceutically acceptable salt or N-oxide thereof” are intended to encompass the compound of formula (I), an N-oxide of formula (I), a pharmaceutically acceptable salt of the compound of formula (I), a solvate of formula (I), or any pharmaceutically acceptable combination of these. Thus by way of non-limiting example used here for illustrative purpose, “a compound of formula (I) or a pharmaceutically acceptable salt thereof” may include a pharmaceutically acceptable salt of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of the formula (I) or pharmaceutically acceptable salt and/or N-oxide thereof.

Particular compounds according to the invention include those mentioned in the examples and their pharmaceutically acceptable N-oxides, salts and solvates.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes enantiomers and diastereoisomers at the attachment point of NR² and R³. The different isomeric forms may be separated or resolved one from the other by conventional methods, or any given isomer may be obtained by conventional synthetic methods or by stereospecific or asymmetric syntheses. Certain compounds of formula (I) may also exist in polymorphic forms and the invention includes such polymorphic forms.

In a further aspect of the invention there is provided a process for preparing compounds of formula (I), and pharmaceutically acceptable salt or N-oxide thereof, which process comprises reacting a compound of formula (II) with a compound of formula (III):

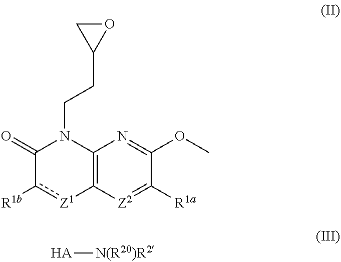

in which:
Z¹ and C(R^{1b}) are linked by a single or a double bond;
R²⁰ is UR⁵ or a group convertible thereto and R^{2′} is R² or a group convertible thereto, wherein Z¹, Z², A, R^{1a}, R^{1b}, R², U and R⁵ are as defined in formula (I) to give a compound of formula (IV):

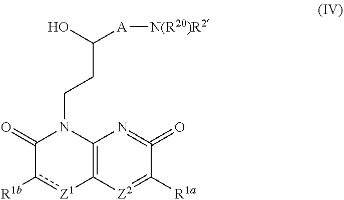

and thereafter cyclising the resulting compound of formula (IV) to give a compound of formula (V):

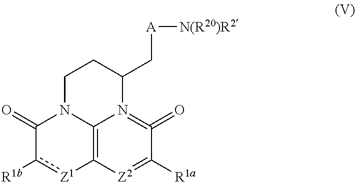

and, optionally or as necessary, oxidising a single bond between Z¹ and C(R^{1b}) to a double bond, converting R²⁰ and R^{2′} to UR⁵ and R², interconverting any variable groups, and/or forming a pharmaceutically acceptable salt or N-oxide thereof.

The reaction between (II) and (III) is an epoxide opening reaction which may be effected by heating in DMF to afford (IV) which can then be cyclised with methanesulphonic anhydride and triethylamine or diisopropylethylamine optionally followed by sodium iodide to give (V). Oxidation of a single bond between Z¹ and C(R^{1b}) may be carried out with 2,3-dichloro-5,6-dicyano-1,4-benzoquinone (DDQ), or if Z¹ is N, the oxidation may be performed with a milder oxidising agent such as manganese dioxide.

Conveniently one of R²⁰ and R^{2′} is an N-protecting group, such as such as t-butoxycarbonyl, benzyloxycarbonyl or 9-fluorenylmethyloxycarbonyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis with, for example trifluoroacetic acid or hydrochloric acid.

The invention further provides compounds of formula (IV) or (V) in which R²⁰ is hydrogen.

The free amine of formula (IV) or (V) in which R²⁰ is hydrogen may be converted to NR²UR⁵ by conventional means such as amide formation with an acyl derivative R⁵COW, for compounds where U is CO or, where U is CH₂, by alkylation with an alkyl halide R⁵CH₂-halide in the presence of base, acylation/reduction with an acyl derivative R⁵COW or reductive alkylation with an aldehyde R⁵CHO under conventional conditions (see for examples Smith, M. B.; March, J. M. Advanced Organic Chemistry, Wiley-Interscience 2001). The appropriate reagents containing the required R⁵ group are known compounds or may be prepared analogously to known compounds, see for example WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2004/035569, WO2004/089947, WO2003082835, WO2002026723, WO06002047, WO06014580, WO06010040, WO06017326, WO06012396, WO06017468, WO06020561, EP0559285, WO07016610, WO07081597, WO07071936, WO07115947, WO07118130, WO07122258, WO06132739, WO06134378, WO06137485, WO06081179, WO06081264, WO06081289, WO06081178, WO06081182, WO08006648, WO08003690, WO08009700, WO2007067511, WO08116815, WO08128953, WO08128942, WO08128962 and WO08128961.

Where R⁵ contains an NH group, this may be protected with a suitable N-protecting group such as t-butoxycarbonyl, benzyloxycarbonyl or 9-fluorenylmethyloxycarbonyl during the coupling of the R⁵ derivative with the free amine of formula (IIB). The protecting group may be removed by conventional methods, such as by treatment with trifluoroacetic acid.

When Z¹ and Z²═CH, and R^{1b}═H and Z¹ and C(R^{1b}) are linked by a single bond, compounds of formula (II) may be prepared by Scheme 1:

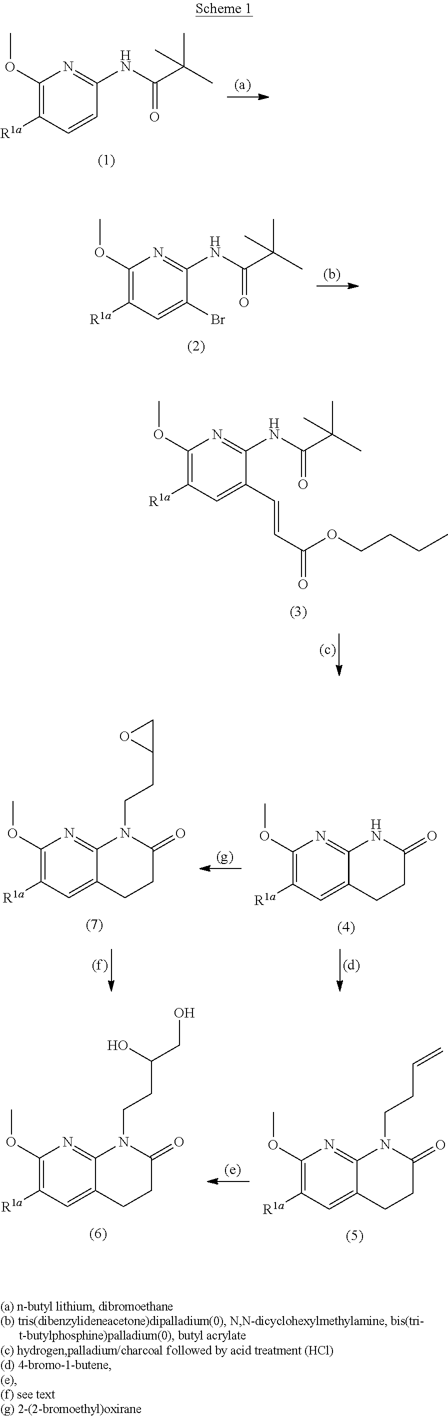

Metallation of (1) (commercially available) with n-butyl lithium followed by bromination with dibromoethane affords bromopyridine derivative (2) (see Zhichkin, P. et al, Synlett (2006) (3) 379-382 for examples of this type of metallation chemistry). Heck reaction of (2) using palladium catalysis (see Sydorenko, N. et al, Organic & Biomolecular Chemistry (2005) 3(11) 2140-2144 for an example of this type of catalysis in a Heck reaction) gives acryate (3). Hydrogenation of the double bond of (3) followed by acid treatment to remove the pivalate residue and effect lactamisation yields the bicyclic lactam (4). Conversion to the epoxide (7) can be effected in a number of ways. For example, reaction with racemic 2-(2-bromoethyl)oxirane under basic conditions affords the epoxide (7), commercially available (2S)-2-(2-bromoethyl)oxirane gives the chiral epoxide. Alternatively, alkylation with 4-bromo-1-butene under basic conditions affords the corresponding N-butenyl material (5) which can be epoxidised under standard achiral or chiral conditions to give the corresponding achiral or chiral epoxides. For example, dihydroxylation can be carried out to give (6) (for example using AD-mix-α or β), which can be converted to (7) under standard conditions, for example by conversion to a mono-tosylate followed by treatment with base.

Compounds of formula (II) in which Z¹ and C(R^{1b}) are linked by a double bond may be prepared from compounds of formula (VI) by Scheme 2:

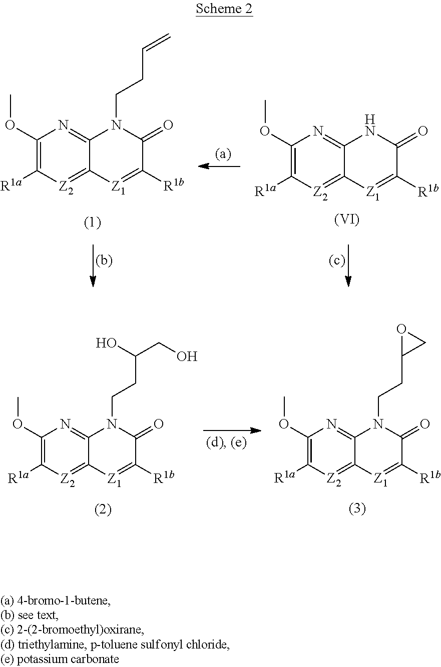

Conversion to the epoxide (3) can be effected in a number of ways—For example, reaction with racemic 2-(2-bromoethyl)oxirane under basic conditions affords the epoxide (3). Commercially available (2S)-2-(2-bromoethyl)oxirane gives the chiral epoxide. Alternatively, alkylation with 4-bromo-1-butene under basic conditions affords the corresponding N-butenyl material (1) which can be converted to the epoxide under standard achiral or chiral conditions. For example, dihydroxylation can be carried out to give (2) (for example using AD-mix-α or β) which can be converted to the epoxide (3) under standard conditions, for example by conversion to a mono-tosylate followed by treatment with base

Compounds of formula (VI) are known compounds or may be prepared analogously to known compounds, see for example WO2006134378, WO2006137485, WO2007138974, WO2008009700 and WO2008128942.

Compounds of formula (VI) where Z¹ is N and Z² is CH may be prepared by Scheme 3:

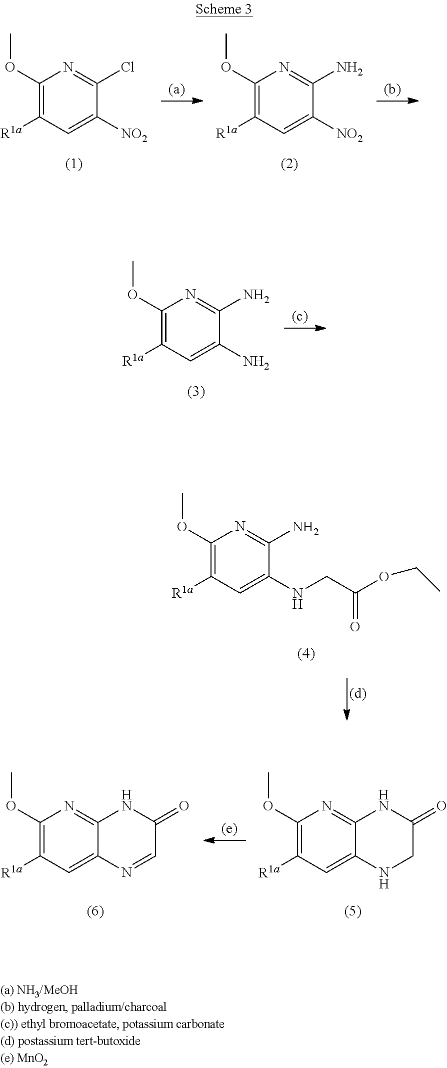

Reaction of nitropyridine (1) with ammonia affords nitro-pyridine (2) which is reduced to bis-aniline (3). Alkylation with ethyl bromoacetate followed by cyclisation with potassium tert-butoxide gives (5). Subsequent oxidation with manganese(II)oxide gives the ring system (6). Alternatively, reaction of (3) with ethyl glyoxylate also gives (6), (for an example of this process see WO2006134378, Example 1(a)).

Compounds of formula (VI) in which Z² is N and Z¹ is CH may be prepared by Scheme 4:

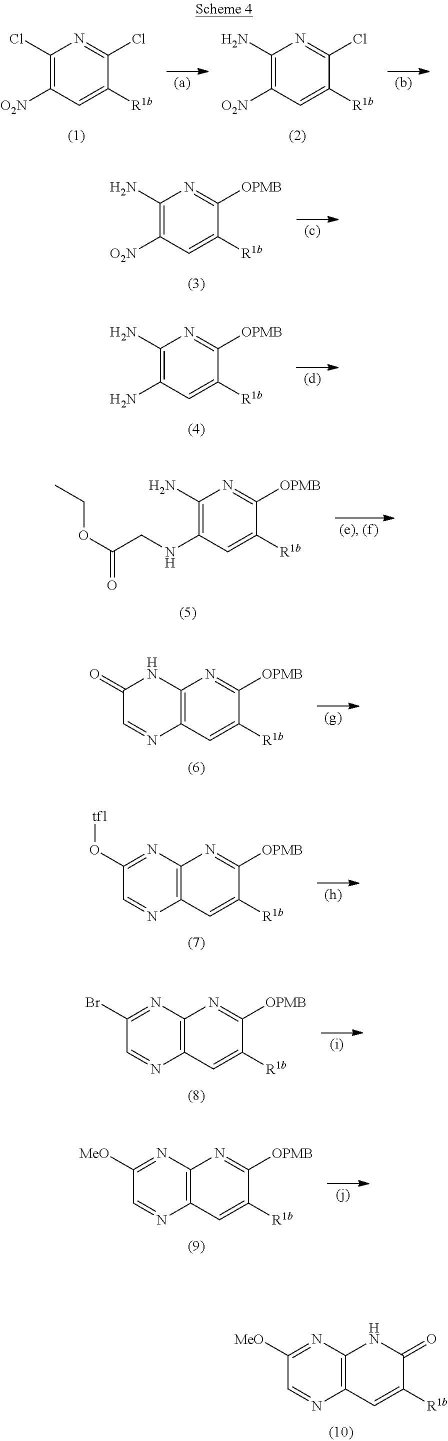

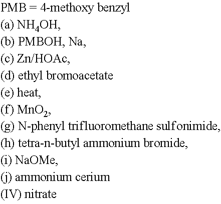

Thus, dichloropyridine (1) can be converted into amino pyridine (2) by treatment with ammonia either with heating or under pressure, optionally in the presence of base, such as sodium carbonate, The 4-methoxybenzyloxy substituent can be introduced by reaction of the chloropyridine with the anion of 4-methoxybenzyl alcohol, for example in the presence of sodium. Reduction of the nitro group can be effected by a number of conditions, (see for examples Smith, M. B.; March, J. M. Advanced Organic Chemistry, Wiley-Interscience 2001), for example sodium dithionite, hydrogenation, or zinc/acetic acid. The diamino compound (4) can be converted to (5) using ethyl bromoacetate, in the presence of base. Cyclisation of the resultant product can be effected by heating, and the ring oxidised by a number of oxidation methods, for example manganese dioxide. Conversion of the pyridazinone ring in (6) to the methoxy pyridazine in (9) can be effected via conversion to the halo pyridazine, for example the bromo pyridazine (8) which can be prepared from the triflate (7) on treatment with tetra-n-butyl ammonium bromide. Removal of the 4-methoxybenzyl protecting group in (9) can be effected using a variety of conditions (for examples see “Protective Groups in Organic Synthesis, T. W. Greene and P. G. M. Wuts, Wiley-Interscience, 1999), for example ammonium cerium(IV) nitrate, to give (10).

Compounds of formula (VI) in which Z¹ and Z² are both N may be prepared by Scheme 5:

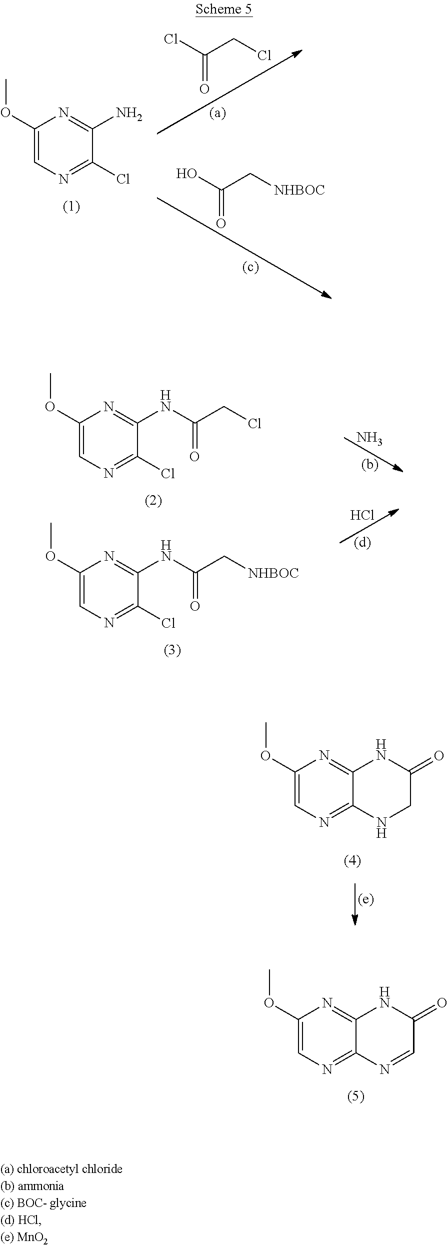

Compound (I) (Bioorganic & Medicinal Chemistry Letters (2005), 15(24), 5446-5449) is converted to (2) by acylation with chloroacetyl chloride followed by treatment with ammonia to give (4). Alternatively (1) may be converted to (3) by coupling with N-{[(1,1-dimethylethyl)oxy]carbonyl}glycine (BOC-glycine) followed by acidic deprotection to give (4).

Interconversions of R^{1a}, R^{1b}, R², A and R⁵ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

Interconversion of R^{1a} and R^{1b} groups may be carried out conventionally, on compounds of formula (I). For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formula (III) HA-N(R²⁰)R^{2′} are known compounds or may be prepared analogously to known compounds, see for example WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2003082835, WO2002026723, WO06002047, WO06014580, WO06134378, WO06137485, WO2007138974 and WO2008009700.

Further details for the preparation of compounds of formula (I) are found in the examples.

The antibacterial compounds according to the invention may be formulated for administration in any convenient way for use in human or veterinary medicine, by analogy with other antibacterials/antitubercular compounds.

The pharmaceutical compositions of the invention may be formulated for administration by any route and include those in a form adapted for oral, topical or parenteral use and may be used for the treatment of bacterial infection including tuberculosis in mammals including humans.

The compositions may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to about 1.5 to about 50 mg/kg per day. Suitably the dosage is from 5 to 30 mg/kg per day.

The compound of formula (I) may be the sole therapeutic agent in the compositions of the invention or a combination with other antibacterials including antitubercular compounds. If the other antibacterial is a β-lactam then a β-lactamase inhibitor may also be employed.

Compounds of formula (I) may be used in the treatment of bacterial infections caused by a wide range of organisms including both Gram-negative and Gram-positive organisms, such as upper and/or lower respiratory tract infections, skin and soft tissue infections and/or urinary tract infections. Compounds of formula (I) may be also used in the treatment of tuberculosis caused by Mycobacterium tuberculosis. Some compounds of formula (I) may be active against more than one organism. This may be determined by the methods described herein.

The following examples illustrate the preparation of certain compounds of formula (I) and the activity of certain compounds of formula (I) against various bacterial organisms including Mycobacterium tuberculosis.

### EXAMPLES AND EXPERIMENTAL

### General

### Abbreviations in the Examples:

MS=mass spectrum
ES=Electrospray mass spectroscopy

### LCMS

RT=room temperature

Certain reagents are also abbreviated herein. TFA refers to trifluoroacetic acid, Pd/C refers to palladium on carbon catalyst, DCM refers to dichloromethane, MeOH refers to methanol, DMF refers to dimethylformamide, EtOH refers to ethanol, NaBH(OAc)₃ refers to sodium triacetoxyborohydride.

Proton nuclear magnetic resonance (¹H NMR) spectra were recorded at 400 or 250 MHz, and chemical shifts are reported in parts per million (ppm) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, td=triplet of doublets, app=apparent, br=broad. J indicates the NMR coupling constant measured in Hertz. CDCl₃ is deuteriochloroform and CD₃OD is tetradeuteriomethanol. Mass spectra were obtained using electrospray (ES) ionization techniques. All temperatures are reported in degrees Celsius.

Amberlyst®A21 is a weakly basic, macroreticular resin with alkyl amine functionality, ®Registered trademark of Rohm & Haas Co.

AD mix alpha can be prepared by mixing potassium osmate (K₂OsO₄.2H₂O) (0.52 g), (3a,9R,3′″a,4′″b,9′″R)-9,9′-[1,4-phthalazinediylbis(oxy)]bis[6′-(methyloxy)-10,11-dihydrocinchonan] [(DHQ)₂PHAL] (5.52 g), then adding potassium ferricyanide [K₃Fe(CN)₆] (700 g) and powdered potassium carbonate (294 g). This mixture is stirred in a blender for 30 minutes. This provides approximately 1 kg of AD mix alpha, which is commercially available from Aldrich. See K. Barry Sharpless et al, J. Org. Chem., 1992, 57 (10), 2771. AD mix beta is the corresponding mixture prepared with (9S,9′″S)-9,9′-[1,4-phthalazinediylbis(oxy)]bis[6′-(methyloxy)-10,11-dihydrocinchonan] [(DHQD)₂PHAL]. Where AD mix alpha/beta is referred to, this is a 1:1 mixture of the alpha and beta mix.

Celite® is a filter aid composed of acid-washed diatomaceous silica, and is a trademark of Manville Corp., Denver, Colo.

SCX Cartridge is an ion exchange column containing strong cation exchange resin (benzene sulfonic acid) supplied by Varian, USA.

Chiralpak AD-H columns comprise of silica for preparative columns (5 um particle size, 21 mm ID×250 mm L) coated with Amylose tris(3,5-dimethylphenylcarbamate) (Chiral Technologies USA). Measured retention times are dependent on the precise conditions of the chromatographic procedures. Where quoted below in the Examples they are indicative of the order of elution.

As will be understood by the skilled chemist, references to preparations carried out in a similar manner to, or by the general method of, other preparations, may encompass variations in routine parameters such as time, temperature, workup conditions, minor changes in reagent amounts etc.

Reactions involving metal hydrides including lithium hydride, lithium aluminium hydride, di-isobutylaluminum hydride, sodium hydride, sodium borohydride and sodium triacetoxyborohydride are carried out under argon or other inert gas.

### Example 1

### 6-([4-[(2,3-Dihydro[1,4]dioxino[2,3-c]pyridin-7-ylmethyl)amino]-1-piperidinyl]methyl)-5,6-dihydro-3H,4H,7H-1,3a,6a-triazaphenalene-3,7-dione dihydrochloride

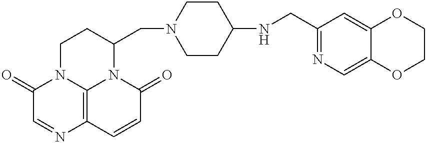

### (a) 4-(3-Buten-1-yl)-6-(methyloxy)pyrido[2,3-b]pyrazin-3(4H)-one

To a stirred solution of 6-(methyloxy)pyrido[2,3-b]pyrazin-3(4H)-one (1.4 g, 7.9 mmole) (for a synthesis see WO2006134378 intermediate 204 or Preparation A below) in DMF (30 mL) under an argon atmosphere was added sodium hydride 60% dispersion in oil (0.35 g, 8.7 mmole) and the mixture stirred at RT for 45 mins. 4-Bromo-1-butene (0.88 mL, 8.7 mmole) was then added and the mixture stirred at RT for a further 18 h. The mixture was then evaporated to dryness and the residue partitioned between water (70 mL) and DCM (2×150 mL). The combined organic layers were dried over Na₂SO₄, filtered and evaporated to dryness. Chromatography on silica gel eluting with a gradient of 0-10% methanol/DCM gave the title compound as a brown solid (1.6 g).

MS (ES+), m/z 232 (MH⁺, 100%).

### (b) 4-(3,4-Dihydroxybutyl)-6-(methyloxy)pyrido[2,3-b]pyrazin-3(4H)-one

To a mixture of 4-(3-buten-1-yl)-6-(methyloxy)pyrido[2,3-b]pyrazin-3(4H)-one (1.6 g, 6.9 mmole) tert-butanol (60 mL) and water (60 mL) was added AD-mix-α (Aldrich) (4.6 g) and AD-mix-β (Aldrich) (4.6 g) and the mixture stirred at RT for 18 h. Water (200 mL) was added and the mixture extracted with 10% methanol/DCM (2×250 mL). The combined organic layers were dried over Na₂SO₄, filtered and evaporated to dryness to give the title compound (1.6 g) as a light brown solid.

MS (ES+), m/z 266 (MH⁺, 30%).

### (c) 2-Hydroxy-4-[6-(methyloxy)-3-oxopyrido[2,3-b]pyrazin-4(3H)-yl]butyl 4-methylbenzenesulfonate

To a stirred suspension of 4-(3,4-dihydroxybutyl)-6-(methyloxy)pyrido[2,3-b]pyrazin-3(4H)-one (1.6 g, 6 mmole) in DCM (100 mL) was added triethylamine (1.25 mL, 9 mmole), p-toluenesulphonyl chloride (1.28 g, 7 mmole) and dibutyltin oxide (0.037 g, 0.12 mmole) and the mixture stirred at RT for 18 h. Water (150 mL) was added and the mixture extracted with DCM (2×150 mL). The combined organics were dried over Na₂SO₄, filtered and evaporated to dryness to give the title compound as a light brown oil (2.67 g).

MS (ES+), m/z 420 (MH⁺, 100%).

### (d) 6-(Methyloxy)-4-[2-(2-oxiranyl)ethyl]pyrido[2,3-b]pyrazin-3(4H)-one

2-Hydroxy-4-[6-(methyloxy)-3-oxopyrido[2,3-b]pyrazin-4(3H)-yl]butyl 4-methylbenzenesulfonate (2.6 g, 6 mmole) was dissolved in methanol (100 mL) and treated with sodium hydrogen carbonate (1.5 g, 17.8 mmole) and stirred at RT for 20 h then heated at 50° C. for 3 h. The mixture was then evaporated to dryness and the residue partitioned between water (50 mL) and DCM (2×75 mL). The combined organics were separated and dried over Na₂SO₄, filtered and evaporated to dryness. The residue was chromatographed twice on silica gel eluting with a gradient of 0-10% methanol/DCM then a gradient of 0-8% methanol/DCM. This gave a mixture containing the title compound as a light brown oil (1.1 g).

MS (ES+), m/z 248 (MH⁺, 70%).

### (e) 1,1-Dimethylethyl (2,3-dihydro[1,4]dioxino[2,3-c]pyridin-7-ylmethyl)(1-{2-hydroxy-4-[6-(methyloxy)-3-oxopyrido[2,3-b]pyrazin-4(3H)-yl]butyl}-4-piperidinyl)carbamate

6-(Methyloxy)-4-[2-(2-oxiranyl)ethyl]pyrido[2,3-b]pyrazin-3(4H)-one (0.28 g, 1.1 mmole) was dissolved in DMF (5 mL) and treated with 1,1-dimethylethyl (2,3-dihydro[1,4]dioxino[2,3-c]pyridin-7-ylmethyl)4-piperidinylcarbamate (0.79 g, 2.2 mmole) (for a synthesis see WO2004058144 Example 99(h)). The mixture was stirred at 130° C. for 1 h then at 120° C. for 18 h. The mixture was then evaporated to dryness and the residue partitioned between water (20 mL) and 10% methanol/DCM (2×20 mL). The combined organics were separated and dried over Na₂SO₄, filtered and evaporated to dryness. The residue was chromatographed on silica gel eluting with a gradient of 0-8% methanol/DCM. This gave the title compound as a light brown oil (0.285 g).

MS (ES+), m/z 597 (MH⁺, 100%).

### (f) 1,1-Dimethylethyl (2,3-dihydro[1,4]dioxino[2,3-c]pyridin-7-ylmethyl){1-[(3,7-dioxo-5,6-dihydro-3H,4H,7H-1,3a,6a-triazaphenalen-6-yl)methyl]-4-piperidinyl}carbamate

1,1-Dimethylethyl (2,3-dihydro[1,4]dioxino[2,3-c]pyridin-7-ylmethyl)(1-{2-hydroxy-4-[6-(methyloxy)-3-oxopyrido[2,3-b]pyrazin-4(3H)-yl]butyl}-4-piperidinyl)carbamate (0.285 g, 0.48 mmole) was dissolved in chloroform (10 mL) and treated with methanesulphonic anhydride (0.166 g, 0.95 mmole) and diisopropylethylamine (0.162 mL, 0.95 mmole). The mixture was heated under reflux with stirring under argon for 18 h, allowed to cool then washed with saturated NaHCO₃ solution (30 mL). The aqueous layer was extracted with DCM (2×30 mL) and the combined organic extracts were dried over Na₂SO₄ filtered and evaporated to dryness. The residue was chromatographed on silica gel eluting with a gradient of 0-12% methanol/DCM. This gave the title compound as a light brown oil (0.166 g).

MS (ES+), m/z 565 (MH⁺, 100%).

### (g) Title Compound

1,1-Dimethylethyl (2,3-dihydro[1,4]dioxino[2,3-c]pyridin-7-ylmethyl){1-[(3,7-dioxo-5,6-dihydro-3H,4H,7H-1,3a,6a-triazaphenalen-6-yl)methyl]-4-piperidinyl}carbamate (0.166 g, 0.29 mmole) was dissolved in DCM (10 mL) and treated with trifluoroacetic acid (2 mL). The mixture was stirred at RT for 18 h then evaporated to dryness. The residue was partitioned between saturated potassium carbonate solution (15 mL) and 10% methanol/DCM (2×20 mL). The combined organic extracts were dried over Na₂SO₄ filtered and evaporated to dryness. The residue was chromatographed on silica gel eluting with a gradient of 2-12% 2M ammonia/methanol in DCM. This gave the free base of the title compound as a light brown oil (0.125 g).

¹H NMR free base (250 MHz, CDCl₃): δ 1.31-1.51 (2H, m), 1.78-2.82 (11H, m), 2.99-3.14 (1H, m), 3.51-3.68 (1H, m), 3.78 (2H, s), 4.23-4.38 (4H, m), 4.45-4.57 (1H, m), 5.07-5.15 (1H, m), 6.44 (1H, d, J 10 Hz), 6.80 (1H, s), 7.73 (1H, d, J 10 Hz), 7.89 (1H, s), 8.09 (1H, s). MS (ES+), m/z 465 (MH⁺, 100%).

The free base of the title compound in methanol/DCM 1:1 (5 mL) was converted to the dihydrochloride salt by addition of excess 4.0M hydrogen chloride in 1,4-dioxane (0.13 mL) then evaporating to dryness to give an off-white solid.

### Example 2

### 6-({4-[([1,3]Oxathiolo[5,4-c]pyridin-6-ylmethyl)amino]-1-piperidinyl}methyl)-5,6-dihydro-3H,4H,7H-1,3a,6a-triazaphenalene-3,7-dione dihydrochloride

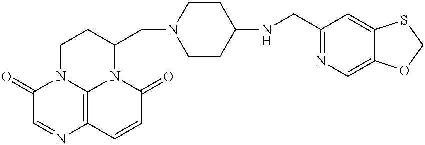

### (a) 1,1-Dimethylethyl (1-{2-hydroxy-4-[6-(methyloxy)-3-oxopyrido[2,3-b]pyrazin-4(3H)-yl]butyl}-4-piperidinyl)carbamate

6-(Methyloxy)-4-[2-(2-oxiranyl)ethyl]pyrido[2,3-b]pyrazin-3(4H)-one (for a preparation see Example 1(d)) (0.78 g, 3.1 mmole) was dissolved in DMF (10 mL) and treated with 1,1-dimethylethyl 4-piperidinylcarbamate (0.95 g, 4.7 mmole). The mixture was heated at 130° C. with stirring under argon for 18 h. The mixture was evaporated to dryness and the residue partitioned between water (50 mL) and ethyl acetate (2×30 mL). The combined organics were washed with more water (30 mL) then saturated sodium chloride solution (30 mL), separated then dried over Na₂SO₄, filtered and evaporated to dryness. The residue was chromatographed on silica gel eluting with a gradient of 2-10% methanol/DCM. This gave the title compound as a cream foam (0.426 g).

MS (ES+), m/z 448 (MH⁺, 100%).

### (b) 1,1-Dimethylethyl {1-[(3,7-dioxo-5,6-dihydro-3H,4H,7H-1,3a,6a-triazaphenalen-6-yl)methyl]-4-piperidinyl}carbamate

1,1-Dimethylethyl (1-{2-hydroxy-4-[6-(methyloxy)-3-oxopyrido[2,3-b]pyrazin-4(3H)-yl]butyl}-4-piperidinyl)carbamate (0.426 g, 0.95 mmole) was dissolved in chloroform (15 mL) and treated with methanesulphonic anhydride (0.32 g, 1.9 mmole) and diisopropylethylamine (0.32 mL, 1.9 mmole). The mixture was heated under reflux with stirring under argon for 18 h, allowed to cool then washed with saturated NaHCO₃ solution (20 mL). The aqueous layer was extracted with DCM (2×30 mL) and the combined organic extracts were dried over Na₂SO₄ filtered and evaporated to dryness. This gave the title compound as a pale orange solid (0.4 g).

MS (ES+), m/z 416 (MH⁺, 50%).

### (c) 6-[(4-Amino-1-piperidinyl)methyl]-5,6-dihydro-3H,4H,7H-1,3a,6a-triazaphenalene-3,7-dione dihydrochloride salt

1,1-Dimethylethyl {1-[(3,7-dioxo-5,6-dihydro-3H,4H,7H-1,3a,6a-triazaphenalen-6-yl)methyl]-4-piperidinyl}carbamate (0.95 mmole) was dissolved in DCM (20 mL) and methanol (20 mL). 4M Hydrogen chloride in 1,4-dioxane (1 mL) was added and the mixture stirred at RT for 30 mins. The mixture was then evaporated to dryness to give the title compound as a light brown solid (0.37 g).

¹H NMR (250 MHz, DMSO-d₆): δ 1.30-4.05 (14H, m), 4.09-4.31 (1H, m), 5.17-5.33 (1H, m), 6.39 (1H, d, J 10 Hz), 7.84 (1H, d, J 10 Hz), 7.86 (1H, s), 8.23-8.55 (3H, brs), 10.49-10.68 (1H, brs).

### (d) Title Compound

A mixture of 6-[(4-amino-1-piperidinyl)methyl]-5,6-dihydro-3H,4H,7H-1,3a,6a-triazaphenalene-3,7-dione dihydrochloride salt (0.25 g, 0.644 mmol) and [1,3]oxathiolo[5,4-c]pyridine-6-carbaldehyde (for a synthesis see WO2004058144 Example 61) (0.108 g, 0.644 mmol) in methanol (20 mL) and acetic acid (0.1 mL) was treated with sodium acetate (0.211 g, 2.58 mmol) and (polystyrylmethyl)trimethylammonium cyanoborohydride (0.966 g, 3.86 mmol) and stirred at RT for 66 h. The resin was filtered off and washed with 1:1 DCM/methanol (3×30 mL). The combined filtrates were evaporated to dryness and the residue was chromatographed on silica gel eluting with 2-12% 2M ammonia/methanol in DCM to give the free base of the title compound as a yellow oil (0.11 g).

¹H NMR free base (250 MHz, CDCl₃): δ 1.31-1.51 (2H, m), 1.79-2.82 (11H, m), 3.03-3.17 (1H, m), 3.55-3.72 (1H, m), 3.81 (2H, s), 4.45-4.58 (1H, m), 5.09-5.19 (1H, m), 5.72 (2H, s), 6.43 (1H, d, J 10 Hz), 7.19 (1H, s), 7.73 (1H, d, J 10 Hz), 7.90 (1H, s), 7.99 (1H, s). MS (ES+), m/z 467 (MH⁺, 100%).

The free base of the title compound was dissolved in methanol (3 mL) and treated with an excess of 4M HCl in 1,4-dioxane and then evaporated to dryness to give the title compound as a pale yellow solid.

### Example 3

### 6-({4-[(2,3-Dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione dihydrochloride

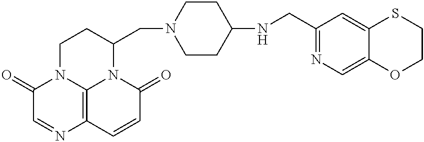

Prepared as in the general method of Example 2(d) from 6-[(4-amino-1-piperidinyl)methyl]-5,6-dihydro-3H,4H,7H-1,3a,6a-triazaphenalene-3,7-dione dihydrochloride salt and 2,3-dihydro[1,4]oxathiino[2,3-c]pyridine-7-carbaldehyde (for a synthesis see WO2004058144 Example 60), to give the free base of the title compound.

¹H NMR free base (250 MHz, CDCl₃): δ 1.29-1.51 (2H, m), 1.78-2.87 (11H, m), 3.03-3.21 (3H, m), 3.53-3.71 (1H, m), 3.75 (2H, s), 4.37-4.41 (2H, m), 4.44-4.61 (1H, s), 5.09-5.19 (1H, m), 6.43 (1H, d, J 10 Hz), 6.99 (1H, s), 7.74 (1H, d, J 10 Hz), 7.91 (1H, s), 8.01 (1H, s). MS (ES+), m/z 481 (MH⁺, 100%).

The free base of the title compound was dissolved in methanol (3 mL) and treated with an excess of 4M HCl in 1,4-dioxane and then evaporated to dryness to give the title compound as a pale yellow solid.

### Example 4

### 6-[(4-{[(3-oxo-3,4-dihydro-2H-pyrido[3,2-b][1,4]thiazin-6-yl)methyl]amino}-1-piperidinyl)methyl]-5,6-dihydro-3H,4H,7H-1,3a,6a-triazaphenalene-3,7-dione dihydrochloride

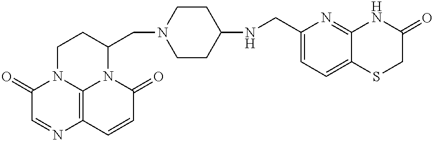

Prepared as in the general method of Example 2(d) from 6-[(4-amino-1-piperidinyl)methyl]-5,6-dihydro-3H,4H,7H-1,3a,6a-triazaphenalene-3,7-dione dihydrochloride salt and 3-oxo-3,4-dihydro-2H-pyrido[3,2-b][1,4]thiazine-6-carbaldehyde (for a synthesis see WO2003087098, Example 301(d)) to give the free base of the title compound.

¹H NMR free base (250 MHz, CDCl₃): δ 1.31-1.51 (2H, m), 1.78-2.83 (11H, m), 3.02-3.18 (1H, m), 3.45 (2H, s), 3.53-3.72 (1H, m), 3.79 (2H, s), 4.45-4.61 (1H, m), 5.08-5.19 (1H, m), 6.45 (1H, d, J 10 Hz), 6.98 (1H, d, J 8 Hz), 7.57 (1H, d, J 8 Hz), 7.77 (1H, d, J 10 Hz), 7.91 (1H, s), 8.15-8.39 (1H, brs). MS (ES+), m/z 494 (MH⁺, 100%).

The free base of the title compound was dissolved in methanol (3 mL) and treated with an excess of 4M HCl in 1,4-dioxane and then evaporated to dryness to give the title compound as a pale yellow solid.

### Example 5A

### (6S)-6-({4-[(2,3-Dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione dihydrochloride

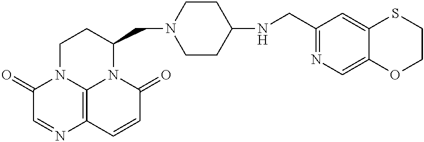

### (a) 6-(Methyloxy)-4-{2-[(2S)-2-oxiranyl]ethyl}pyrido[2,3-b]pyrazin-3(4H)-one

To a solution of 6-(methyloxy)pyrido[2,3-b]pyrazin-3(4H)-one (for a synthesis see WO2006134378 intermediate 204 or Preparation A below) (15 g, 80 mmol) in DMF (200 ml) under argon was added sodium hydride 60% dispersion in oil (3.54 g, 88 mmol) and the mixture stirred at RT for 1 h. (2S)-2-(2-Bromoethyl)oxirane (8.78 ml, 88 mmol) was then added and the mixture stirred at RT for 18 h. The DMF was then evaporated and the residue partitioned between ethyl acetate (400 ml) and water (400 ml). the organic layer was separated and washed with more water (2×400 ml) then dried over Na₂SO₄, filtered and evaporated to dryness to give the title compound as a brown solid (19.19 g).

MS (ES+), m/z 248 (MH⁺, 100%).

### (b) 1,1-Dimethylethyl (1-{(2S)-2-hydroxy-4-[6-(methyloxy)-3-oxopyrido[2,3-b]pyrazin-4(3H)-yl]butyl}-4-piperidinyl)carbamate

6-(Methyloxy)-4-{2-[(2S)-2-oxiranyl]ethyl}pyrido[2,3-b]pyrazin-3(4H)-on (19.19 g, 69.9 mmol) and 1,1-dimethylethyl 4-piperidinylcarbamate (18.19 g, 91 mmol) were dissolved/suspended in DMF (200 ml) then heated at 130° C. with stirring under argon for 18 h. The mixture was then evaporated to dryness and the residue dissolved in DCM (300 mL) then chromatographed on silica gel eluting with 4% methanol/DCM. This gave the title compound as a dark brown oil (28.7 g).

MS (ES+), m/z 448 (MH⁺, 100%).

### (c) 1,1-Dimethylethyl (1-{[(6S)-3,7-dioxo-5,6-dihydro-3H,4H,7H-1,3a,6a-triazaphenalen-6-yl]methyl}-4-piperidinyl)carbamate

1,1-Dimethylethyl (1-{(2S)-2-hydroxy-4-[6-(methyloxy)-3-oxopyrido[2,3-b]pyrazin-4(3H)-yl]butyl}-4-piperidinyl)carbamate (28.7 g, 54.5 mmol) was dissolved in chloroform (900 ml) with stirring and treated with diisopropylethylamine (19.04 ml, 109 mmol) and methanesulfonic anhydride (18.99 g, 109 mmol). The mixture was then heated under reflux under argon for 18 h. The mixture was allowed to cool and then washed with saturated sodium NaHCO₃ solution (2×600 mL). The aqueous washings were then extracted with 10% methanol/DCM (2×600 mL). The organic extracts were then combined and washed with more saturated sodium hydrogen carbonate solution (1000 mL), separated then dried over Na₂SO₄, filtered and evaporated to dryness to give a brown waxy solid. This was triturated with diethyl ether (600 mL) to give a solid which was filtered off and dried under vacuum. This gave the title compound as a light brown solid (14.51 g).

MS (ES+), m/z 416 (MH⁺, 100%).

### (d) (6S)-6-[(4-Amino-1-piperidinyl)methyl]-5,6-dihydro-3H,4H,7H-1,3a,6a-triazaphenalene-3,7-dione dihydrochloride salt

1,1-Dimethylethyl (1-{[(6S)-3,7-dioxo-5,6-dihydro-3H,4H,7H-1,3a,6a-triazaphenalen-6-yl]methyl}-4-piperidinyl)carbamate (14.5 g, 31.4 mmol) was dissolved/suspended in 1,4-dioxane (400 ml) and 4M hydrogen chloride in 1,4-dioxane (31.4 ml, 126 mmol) added and the mixture stirred at RT for 5 days. The mixture had set solid and was suspended in DCM (400 ml) then evaporated to dryness. The resulting solid was dried at 35° C. in a vacuum oven for 3 days. This gave the title compound as a brown solid (14.2 g).

¹H NMR (250 MHz, DMSO-d₆): δ 1.99-2.21 (4H, m), 2.89-3.99 (10H, m), 4.23-4.39 (1H, m), 5.15-5.33 (1H, m), 6.39 (1H, d, J 10 Hz), 7.85 (1H, d, J 10 Hz), 7.87 (1H, s), 8.31-8.51 (3H, brs), 10.59-10.73 (1H, brs).

### (e) Title Compound

A mixture of (6S)-6-[(4-amino-1-piperidinyl)methyl]-5,6-dihydro-3H,4H,7H-1,3a,6a-triazaphenalene-3,7-dione dihydrochloride salt (2 g, 5.15 mmol) and 2,3-dihydro[1,4]oxathiino[2,3-c]pyridine-7-carbaldehyde (for a synthesis see WO2004058144 Example 60) (0.747 g, 4.12 mmol) in methanol (200 ml) and acetic acid (1.000 ml) was treated with sodium acetate (0.845 g, 10.30 mmol) and (polystyrylmethyl)trimethylammonium cyanoborohydride (7.73 g, 30.9 mmol) and stirred at RT for 18 h. DCM (200 ml) was added and the mixture filtered to remove the resin washing with 1:1 DCM/methanol (3×50 ml). The combined filtrate and washings were evaporated to dryness. The residue was partitioned between 10% methanol/DCM (250 ml) and saturated NaHCO₃ (300 ml) and the organic layer separated. The aqueous layer was extracted with more 10% methanol/DCM (4×150 ml) and the combined organics were dried over Na₂SO₄ filtered and evaporated to dryness. The residue obtained was chromatographed on silica gel eluting with a gradient of 0-10% 2M ammonia/methanol in DCM. This gave the free base of the title compound as a pale yellow foam (1.1 g).

¹H NMR free base (250 MHz, CDCl₃): δ 1.29-1.51 (2H, m), 1.78-2.87 (11H, m), 3.03-3.21 (3H, m), 3.53-3.71 (1H, m), 3.75 (2H, s), 4.37-4.41 (2H, m), 4.44-4.61 (1H, s), 5.09-5.19 (1H, m), 6.43 (1H, d, J 10 Hz), 6.99 (1H, s), 7.74 (1H, d, J 10 Hz), 7.91 (1H, s), 8.01 (1H, s).

MS (ES+), m/z 481 (MH⁺, 75%).

The free base of the title compound was dissolved in methanol (30 ml) and treated with excess 4.0M hydrogen chloride in 1,4-dioxane (1.2 ml). The resulting solid was filtered off and dried under vacuum to give the title compound as a pale orange solid (0.99 g).

### Example 5B

### (6S)-6-({4-[(2,3-Dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione mono benzoic acid salt

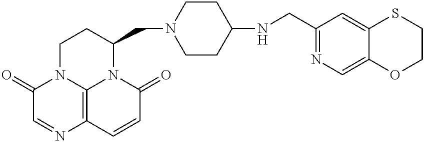

The free base of the title compound (Example 5A(e)) was converted to the monobenzoic acid salt in methanol. The solvent was evaporated to give the title compound as an off-white solid.

### Example 6

### (6S)-6-({4-[(3,4-Dihydro-2H-pyrano[2,3-c]pyridin-6-ylmethyl)amino]-1-piperidinyl}methyl)-5,6-dihydro-3H,4H,7H-1,3a,6a-triazaphenalene-3,7-dione dihydrochloride

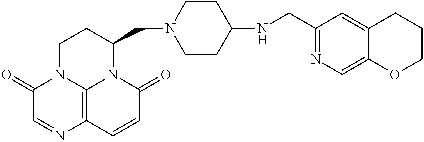

Prepared as in the general method of Example 5A(e) from (6S)-6-[(4-amino-1-piperidinyl)methyl]-5,6-dihydro-3H,4H,7H-1,3a,6a-triazaphenalene-3,7-dione dihydrochloride salt and 3,4-dihydro-2H-pyrano[2,3-c]pyridine-6-carbaldehyde (for a synthesis see WO2004058144 Example 126(e)) to give the free base of the title compound.

¹H NMR of free base (250 MHz, CDCl₃): δ 1.31-1.51 (2H, m), 1.75-2.85 (15H, m), 3.02-3.18 (1H, m), 3.53-3.72 (1H, m), 3.79 (1H, s), 4.19 (2H, t, J 6 Hz), 4.45-4.59 (1H, m), 5.09-5.19 (1H, m), 6.42 (1H, d, J 10 Hz), 6.95 (1H, s), 7.72 (1H, d, J 10 Hz), 7.89 (1H, s), 8.08 (1H, s). MS (ES+), m/z 463 (MH⁺, 40%).

The free base of the title compound was dissolved in methanol (3 ml) and treated with 4.0M hydrogen chloride in 1,4-dioxane (0.085 ml). The resulting solution was evaporated to dryness to give the title compound as an off-white solid.

### Example 7A

### (6S)-6-({4-[([1,3]Oxathiolo[5,4-c]pyridin-6-ylmethyl)amino]-1-piperidinyl}methyl)-5,6-dihydro-3H,4H,7H-1,3a,6a-triazaphenalene-3,7-dione dihydrochloride

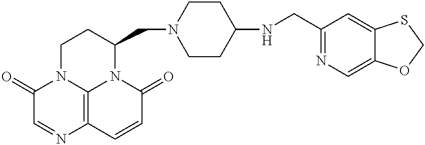

Prepared as in the general method of Example 5A(e) from (6S)-6-[(4-amino-1-piperidinyl)methyl]-5,6-dihydro-3H,4H,7H-1,3a,6a-triazaphenalene-3,7-dione dihydrochloride salt and [1,3]oxathiolo[5,4-c]pyridine-6-carbaldehyde (for a synthesis see WO2004058144 Example 61) to give the free base of the title compound.

¹H NMR of free base (250 MHz, CDCl₃): δ 1.31-1.51 (2H, m), 1.79-2.82 (11H, m), 3.03-3.17 (1H, m), 3.55-3.72 (1H, m), 3.81 (2H, s), 4.45-4.58 (1H, m), 5.09-5.19 (1H, m), 5.72 (2H, s), 6.43 (1H, d, J 10 Hz), 7.19 (1H, s), 7.73 (1H, d, J 10 Hz), 7.90 (1H, s), 7.99 (1H, s). MS (ES+), m/z 467 (MH⁺, 40%).

The free base of the title compound was dissolved in methanol (30 ml) and treated with excess 4.0M hydrogen chloride in 1,4-dioxane (1.2 ml). The resulting solid was filtered off and dried under vacuum to give the title compound.

### Example 7B

### (6S)-6-({4-[([1,3]Oxathiolo[5,4-c]pyridin-6-ylmethyl)amino]-1-piperidinyl}methyl)-5,6-dihydro-3H,4H,7H-1,3a,6a-triazaphenalene-3,7-dione mono benzoic acid salt

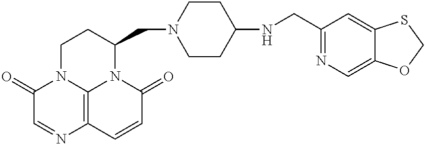

The free base of the title compound (Example 7A) was converted to the monobenzoic acid salt in methanol. The solvent was evaporated to give the title compound as an off-white solid.

### Example 8

### 4-({4-[([1,3]Oxathiolo[5,4-c]pyridin-6-ylmethyl)amino]-1-piperidinyl}methyl)-5,6-dihydro-3H,4H,7H-1,3a,6a-triazaphenalene-3,7-dione dihydrochloride

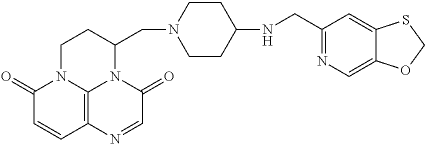

### (a) 5-(3-Buten-1-yl)-3-(methyloxy)pyrido[2,3-b]pyrazin-6(5H)-one

To a solution of 3-(methyloxy)pyrido[2,3-b]pyrazin-6(5H)-one (for a synthesis see WO08009700 Example 94(i)) (1.13 g, 4.46 mmol) in DMF (50 ml) was added sodium hydride 60% dispersion in oil (0.214 g, 5.36 mmol) and the mixture stirred at RT for 1 h. 4-Bromo-1-butene (0.544 ml, 5.36 mmol) was then added and the mixture stirred at RT for 3 h. The DMF was removed under vacuum and the residue partitioned between water (50 mL) and ethyl acetate (2×50 mL). The combined organics were then washed with saturated brine (50 mL) separated and dried over Na₂SO₄, filtered and evaporated to dryness. The residue was purified on silica gel eluting with 0-10% methanol/DCM then chromatographed again on silica gel eluting with 20-70% ethyl acetate/40-60 pet-ether to give after evaporation a mixture containing the title compound as a pale yellow oil (0.76 g).

MS (ES+), m/z 232 (MH⁺, 100%).

### (b) 5-(3,4-dihydroxybutyl)-3-(methyloxy)pyrido[2,3-b]pyrazin-6(5H)-one

5-(3-Buten-1-yl)-3-(methyloxy)pyrido[2,3-b]pyrazin-6(5H)-one (0.76 g, 3.29 mmol) was dissolved in tert-butanol (30 ml) and water (30.0 ml) and treated with AD mix α (2.2 g, 3.29 mmol) and AD mix β (2.2 g, 3.29 mmol) and stirred at RT for 18 h. Water (80 mL) was added and the mixture extracted with 10% methanol/DCM (3×70 mL). The combined organic extracts were dried over Na₂SO₄ filtered and evaporated to dryness to give a pale yellow oil. Trituration with diethyl ether (30 mL) gave a solid which was filtered off and dried under vacuum. This gave the title compound as an off-white solid (0.514 g).

MS (ES+), m/z 288 (M+Na, 100%).

### (c) 2-Hydroxy-4-[3-(methyloxy)-6-oxopyrido[2,3-b]pyrazin-5(6H)-yl]butyl 4-methylbenzenesulfonate

To a solution/suspension of 5-(3,4-dihydroxybutyl)-3-(methyloxy)pyrido[2,3-b]pyrazin-6(5H)-one (0.51 g, 1.923 mmol) in DCM (50 ml) was added triethylamine (0.295 ml, 2.115 mmol), p-toluenesulfonyl chloride (0.403 g, 2.115 mmol) and dibutyltin oxide (9.57 mg, 0.038 mmol) and the mixture stirred at RT for 66 h. The mixture was then washed with water (50 mL) and the organic layer separated and dried over Na₂SO₄, filtered and evaporated to dryness. This gave a mixture as a colourless oil containing the title compound (0.84 g).

MS (ES+), m/z 420 (MH⁺, 100%).

### (d) 3-(Methyloxy)-5-[2-(2-oxiranyl)ethyl]pyrido[2,3-b]pyrazin-6(5H)-one

To a stirred solution/suspension of 2-hydroxy-4-[3-(methyloxy)-6-oxopyrido[2,3-b]pyrazin-5(6H)-yl]butyl 4-methylbenzenesulfonate (0.84 g, 1.702 mmol) in methanol (70 ml) was added NaHCO₃ (0.5 g, 5.95 mmol) and the mixture heated at 45° C. for 18 h. The mixture was then evaporated to dryness and the residue partitioned between water (50 mL) and DCM (2×50 mL). The combined organic layers were dried over Na₂SO₄, filtered and evaporated to dryness. The residue was chromatographed on silica gel eluting with 0-10% methanol/DCM. This gave a mixture containing the title compound as a colourless oil (0.347 g).

MS (ES+), m/z 270 (M+Na, 100%).

### (e) 1,1-Dimethylethyl (1-{2-hydroxy-4-[3-(methyloxy)-6-oxopyrido[2,3-b]pyrazin-5(6H)-yl]butyl}-4-piperidinyl)carbamate

To a stirred solution of 3-(methyloxy)-5-[2-(2-oxiranyl)ethyl]pyrido[2,3-b]pyrazin-6(5H)-one (0.347 g, 0.982 mmol) in DMF (10 ml) was added 1,1-dimethylethyl 4-piperidinylcarbamate (0.295 g, 1.474 mmol) and the mixture heated at 130 C for 18 h under argon. The DMF was then removed under vacuum and the residue partitioned between water (50 mL) and 10% methanol/DCM (2×50 mL). The combined organic extracts were dried over Na₂SO₄ filtered and evaporated to dryness. The residue was chromatographed on silica gel eluting with 0-10% methanol/DCM. This gave the title compound as a pale yellow oil (0.19 g).

MS (ES+), m/z 448 (MH⁺, 100%).

### (f) 1,1-Dimethylethyl {1-[(3,7-dioxo-5,6-dihydro-3H,4H,7H-1,3a,6a-triazaphenalen-4-yl)methyl]-4-piperidinyl}carbamate

To a stirred solution of 1,1-dimethylethyl (1-{2-hydroxy-4-[3-(methyloxy)-6-oxopyrido[2,3-b]pyrazin-5(6H)-yl]butyl}-4-piperidinyl)carbamate (0.19 g, 0.425 mmol) in chloroform (10 ml) was added diisopropylethylamine (0.148 ml, 0.849 mmol) and methanesulfonic anhydride (0.148 g, 0.849 mmol) and the mixture heated under reflux for 18 h. The mixture was allowed to cool and washed with saturated NaHCO₃ solution (30 mL). The organic layer was separated and dried over Na₂SO₄, filtered and evaporated to dryness. Chromatography on silica gel eluting with 0-10% methanol/DCM, followed by a second column eluting with 2-6% methanol/DCM gave the title compound as a yellow oil (0.056 g).

MS (ES+), m/z 416 (MH⁺, 40%).

### (g) 4-[(4-Amino-1-piperidinyl)methyl]-5,6-dihydro-3H,4H,7H-1,3a,6a-triazaphenalene-3,7-dione trifluoroacetate salt

To a solution of 1,1-dimethylethyl {1-[(3,7-dioxo-5,6-dihydro-3H,4H,7H-1,3a,6a-triazaphenalen-4-yl)methyl]-4-piperidinyl}carbamate (0.056 g, 0.135 mmol) in DCM (10 ml) was added TFA (2.0 mL, 26.0 mmol) and the mixture stirred at RT for 18 h. The mixture was then evaporated to dryness and the residue dissolved in methanol (20 mL) and treated with Amberlyst A-21 ion exchange resin (2 g) and stirred at RT for 30 mins. The resin was filtered off and the filtrate evaporated to dryness to give the title compound as an off-white solid (0.058 g). MS (ES+), m/z 316 (MH⁺, 100%).

### (h) Title Compound

To a stirred solution of 4-[(4-amino-1-piperidinyl)methyl]-5,6-dihydro-3H,4H,7H-1,3a,6a-triazaphenalene-3,7-dione trifluoroacetate salt (0.058 g, 0.135 mmol) and [1,3]oxathiolo[5,4-c]pyridine-6-carbaldehyde (for a synthesis see WO2004058144 Example 61) (0.018 g, 0.108 mmol) in methanol (10 ml) and acetic acid (0.05 ml) was added sodium acetate (0.044 g, 0.540 mmol) and (polystyrylmethyl)trimethylammonium cyanoborohydride (0.203 g, 0.810 mmol) and the mixture stirred at RT for 90 h. The resin was filtered off and washed with 1:1 DCM/methanol (3×30 mL). The combined filtrates were evaporated to dryness and the residue was purified on silica gel eluting with 2-12% 2M ammonia/methanol in DCM to give the free base of the title compound as a yellow oil (0.046 g).

¹H NMR of free base (250 MHz, CDCl₃): δ 1.31-1.52 (2H, m), 1.79-2.84 (11H, m), 3.01-3.18 (1H, m), 3.51-3.67 (1H, m), 3.8 (2H, s), 4.52-4.67 (1H, s), 4.97-5.11 (1H, m), 5.71 (2H, s), 6.48 (1H, d, J 10 Hz), 7.18 (1H, s), 7.75 (1H, d, J 10 Hz), 7.89 (1H, s), 8.01 (1H, s).

MS (ES+), m/z 467 (MH⁺, 100%).

The free base of the title compound was dissolved in methanol (3 mL) and treated with an excess of 4M HCl in 1,4-dioxane and then evaporated to dryness to give the dihydrochloride of the product as a pale yellow solid (0.053 g).

### Example 9

### (4S)-4-({4-[(2,3-Dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione dihydrochloride

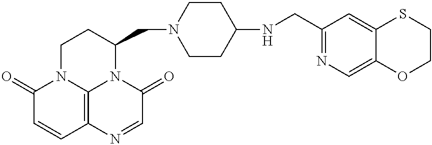

### (a) 3-(Methyloxy)-5-{2-[(2S)-2-oxiranyl]ethyl}pyrido[2,3-b]pyrazin-6(5H)-one

To a solution of 3-(methyloxy)pyrido[2,3-b]pyrazin-6(5H)-one (for a synthesis see WO08009700 Example 94(i)) (3 g, 11.01 mmol) in DMF (100 ml) was added sodium hydride 60% dispersion in oil (0.440 g, 11.01 mmol) and the mixture stirred for 1 h under argon at RT. (2S)-2-(2-Bromoethyl)oxirane (1.2 ml, 12.08 mmol) was then added and the mixture stirred at RT for 3 h. The DMF was removed on a rotary evaporator and the residue partitioned between water (100 ml) and ethyl acetate (2×100 ml). The combined organics were separated and dried over Na₂SO₄, filtered and evaporated to dryness. Chromatography on silica gel eluting with a gradient of 10-100% ethyl acetate/40-60 petroleum ether gave the title compound as an orange oil (0.86 g).

MS (ES+), m/z 248 (MH⁺, 80%).

### (b) 1,1-Dimethylethyl (1-{(2S)-2-hydroxy-4-[3-(methyloxy)-6-oxopyrido[2,3-b]pyrazin-5(6H)-yl]butyl}-4-piperidinyl)carbamate

To a stirred solution of 3-(methyloxy)-5-{2-[(2S)-2-oxiranyl]ethyl}pyrido[2,3-b]pyrazin-6(5H)-one (0.86 g, 2.261 mmol) in DMF (20 ml) was added 1,1-dimethylethyl 4-piperidinylcarbamate 0.86 g, 4.29 mmol) and the mixture heated at 130° C. for 18 h under argon. The mixture was evaporated to dryness and chromatographed on silica gel eluting with a gradient of 0-10% methanol/DCM then re-chromatographed on silica gel with a gradient of 4-16% methanol/DCM. This gave the title compound as a cream foam (0.22 g). MS (ES+), m/z 448 (MH⁺, 100%).

### (c) 1,1-Dimethylethyl (1-{[(4S)-3,7-dioxo-5,6-dihydro-3H,4H,7H-1,3a,6a-triazaphenalen-4-yl]methyl}-4-piperidinyl)carbamate

A stirred solution of 1,1-dimethylethyl (1-{(2S)-2-hydroxy-4-[3-(methyloxy)-6-oxopyrido[2,3-b]pyrazin-5(6H)-yl]butyl}-4-piperidinyl)carbamate (0.22 g, 0.418 mmol) in chloroform (15 ml) was treated with diisopropylethylamine (0.146 ml, 0.836 mmol) and methanesulphonic anhydride (0.146 g, 0.836 mmol) and heated under reflux under argon for 20 h. The mixture was allowed to cool and saturated NaHCO₃ solution (15 ml) added. This mixture was then extracted with 10% methanol/DCM (2×20 ml) and the combined organics dried over Na₂SO₄, filtered and evaporated to dryness to give the title compound as a brown solid (0.24 g).

MS (ES+), m/z 416 (MH⁺, 60%).

### (d) (4S)-4-[(4-Amino-1-piperidinyl)methyl]-5,6-dihydro-3H,4H,7H-1,3a,6a-triazaphenalene-3,7-dione dihydrochloride salt

To a solution of 1,1-dimethylethyl (1-{[(4S)-3,7-dioxo-5,6-dihydro-3H,4H,7H-1,3a,6a-triazaphenalen-4-yl]methyl}-4-piperidinyl)carbamate (0.24 g, 0.404 mmol) in chloroform (2 ml) and methanol (2 ml) was added 4.0M hydrogen chloride in 1,4-dioxane (2 ml, 8.00 mmol) and the mixture stirred at RT for 20 h. The mixture was then evaporated to dryness to give the title compound as a brown solid (0.22 g).

MS (ES+), m/z 316 (MH⁺, 100%).

### (e) Title Compound

To a stirred solution/suspension of (4S)-4-[(4-amino-1-piperidinyl)methyl]-5,6-dihydro-3H,4H,7H-1,3a,6a-triazaphenalene-3,7-dione (0.22 g, 0.397 mmol) and 2,3-dihydro[1,4]oxathiino[2,3-c]pyridine-7-carbaldehyde (for a synthesis see WO2004058144 Example 60) (0.065 g, 0.357 mmol) in chloroform (10 ml) and methanol (1 ml) was added triethylamine (0.166 ml, 1.190 mmol) and the mixture stirred at RT under argon for 1 h. Sodium triacetoxyborohydride (0.252 g, 1.190 mmol) was then added and the mixture stirred for a further 3 h. More aldehyde (0.015 g) and more sodium triacetoxyborohydride (0.084 g) was added and the mixture was stirred at RT for 66 h. Saturated aq NaHCO₃ solution (30 ml) was added and the mixture extracted with 20% methanol/DCM (2×30 mL). The combined organic layers were dried over Na₂SO₄, filtered and evaporated to dryness, then chromatographed on silica gel eluting with a gradient of 0-12% 2M NH₃/MeOH/DCM. This gave the free base of the title compound as a light brown oil (0.128 g).

¹H NMR free base (250 MHz, CDCl₃): δ 1.31-1.53 (2H, m), 1.77-2.82 (11H, m), 3.01-3.14 (1H, m), 3.15-3.22 (2H, m), 3.51-3.69 (1H, m), 3.72 (2H, s), 4.37-4.44 (2H, m), 4.52-4.65 (1H, m), 4.98-5.11 (1H, m), 6.47 (1H, d, J 10 Hz), 6.99 (1H, s), 7.75 (1H, d, J 10 Hz), 7.88 (1H, s), 8.01 (1H, s).

MS (ES+), m/z 481 (MH⁺, 100%).

The free base of the title compound was dissolved in 1:1 methanol/DCM and treated with excess 4M HCl/1,4-dioxane then evaporated to dryness to give the dihydrochloride as a pale orange solid.

### Example 10

### 5-({4-[(2,3-Dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione hydrochloride

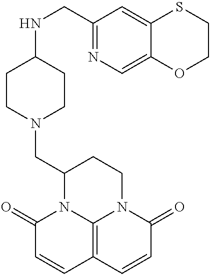

### (a) 1-(3-Buten-1-yl)-7-(methyloxy)-3,4-dihydro-1,8-naphthyridin-2(1H)-one

7-(Methyloxy)-3,4-dihydro-1,8-naphthyridin-2(1H)-one (for a synthesis see WO2008128942 Example 5A(e)) (4 g, 22.5 mmol) was dissolved in dry DMF (100 ml) and treated with sodium hydride 60% dispersion in oil (0.99 g, 24.75 mmol) portionwise. The mixture was stirred for 1 hr at RT under an argon atmosphere. 4-Bromo-1-butene (2.5 ml, 24.75 mmol) was added and the reaction mixture stirred overnight at RT. The DMF was evaporated under reduced pressure and the residue partitioned between water (100 ml) and DCM (100 ml). The aqueous layer was extracted with DCM (3×100 ml). The combined organics were dried over Na₂SO₄ filtered and evaporated to dryness. The residue was chromatographed on silica gel eluting with a gradient of 0-10% methanol/DCM to give the title compound as pale yellow oil (3.9 g, 75%).

MS (ES+) m/z 233 (MH⁺, 50%).

### (b) 1-(3,4-Dihydroxybutyl)-7-(methyloxy)-3,4-dihydro-1,8-naphthyridin-2(1H)-one

1-(3-Buten-1-yl)-7-(methyloxy)-3,4-dihydro-1,8-naphthyridin-2(1H)-one (3.0 g, 12.9 mmol) was dissolved in tert butanol (75 ml) and water (75 ml). After 10 mins AD-mix-α (8.76 g) and AD-mix-β (8.76 g) were added and the mixture stirred overnight at RT. Water (100 ml) was added and the mixture was extracted with 10% methanol/DCM (2×100 ml). The combined organics were dried over Na₂SO₄, filtered and concentrated in vacuo. to give the title compound as a brown oil. (3.34 g, 97%).

MS (ES+) m/z 267 (MH⁺, 100%).

### (c) 2-Hydroxy-4-[7-(methyloxy)-2-oxo-3,4-dihydro-1,8-naphthyridin-1(2H)-yl]butyl 4-methylbenzenesulfonate

1-(3,4-Dihydroxybutyl)-7-(methyloxy)-3,4-dihydro-1,8-naphthyridin-2(1H)-one (3.34 g, 12.6 mmol) was dissolved in DCM and treated with triethylamine (2.63 ml, 18.9 mmol). After 5 mins p-toluenesulphonyl chloride (2.39 g, 12.6 mmol) was added followed by dibutyltin oxide (0.0062 g, 0.25 mmol) and the mixture stirred at RT under argon atmosphere for 5 days. Water (200 ml) was added and the mixture extracted with DCM (2×100 ml). The combined organics were dried over Na₂SO₄, filtered and concentrated in vacuo to give the title compound as a brown oil. (5.51 g).

MS (ES+) m/z 421 (MH⁺, 100%).

### (d) 7-(Methyloxy)-1-[2-(2-oxiranyl)ethyl]-3,4-dihydro-1,8-naphthyridin-2(1H)-one

2-Hydroxy-4-[7-(methyloxy)-2-oxo-3,4-dihydro-1,8-naphthyridin-1(2H)-yl]butyl 4-methylbenzenesulfonate (8.4 g, 16.18 mmol) was dissolved in methanol (400 ml) and treated with potassium carbonate (4.7 g, 32.4 mmol) and stirred for 1 h. The mixture was concentrated in vacuo and the residue partitioned between water/DCM. The mixture was extracted with DCM (3×100 ml)and the combined organics dried over Na₂SO₄, filtered and concentrated in vacuo. The residue was chromatographed on silica gel eluting with a gradient of 0-65% ethyl acetate/hexane to give the title compound as colourless oil. (2.45 g, 60%).

MS (ES+) m/z 249 (MH⁺, 100%)

### (e) 1,1-Dimethylethyl (1-{2-hydroxy-4-[7-(methyloxy)-2-oxo-3,4-dihydro-1,8-naphthyridin-1(2H)-yl]butyl}-4-piperidinyl)carbamate

7-(Methyloxy)-1-[2-(2-oxiranyl)ethyl]-3,4-dihydro-1,8-naphthyridin-2(1H)-one (3.45 g, 13.9 mmol) was dissolved in DMF (100 ml) and treated with 1,1-dimethylethyl 4-piperidinylcarbamate (3.09 g, 15.45 mmol) was added and the reaction mixture heated at 125° C. overnight. The DMF was removed in-vacuo and the residue partitioned between water (200 ml) and 10% methanol/DCM (200 ml). The aqueous layer was extracted with more 10% methanol/DCM (3×100 ml) and the combined organics dried over Na₂SO₄, filtered and concentrated in vacuo. The residue was chromatographed on silica gel eluting with a gradient of 0-10% methanol/DCM to give the title compound as a brown gum (3.1 g 53%).

MS (ES+) m/z 449 (MH⁺, 100%).

### (f) 1,1-Dimethylethyl {1-[(3,9-dioxo-2,3,6,7-tetrahydro-1H,5H,9H-pyrimido[1,2,3-ij]-1,8-naphthyridin-7-yl)methyl]-4-piperidinyl}carbamate

1,1-Dimethylethyl (1-{2-hydroxy-4-[7-(methyloxy)-2-oxo-3,4-dihydro-1,8-naphthyridin-1(2H)-yl]butyl}-4-piperidinyl)carbamate (3.1 g, 6.91 mmol) was dissolved in chloroform (100 ml) and treated with methanesulfonic anhydride (2.89 g, 16.59 mmol) and diisopropylethylamine (2.97 ml, 16.59 mmol). The mixture was heated under reflux overnight. Sodium iodide (1.036 g, 6.91 mmol) was added and the mixture stirred at RT under argon atmosphere then at 40° C. for 5 h. The mixture was allowed to cool then partitioned between water (100 ml) and 10% methanol/DCM (100 ml). The mixture was extracted with more 10% methanol/DCM (4×50 ml) and the combined organics were dried over Na₂SO₄ filtered and concentrated in vacuo to give a brown gum. This was dissolved in acetonitrile and treated with sodium iodide (2.0 g, 13.82 mmol). The mixture was heated at 50° C. for 4 h then partitioned between water (100 ml) and 10% methanol/DCM (100 ml). The aqueous layer was extracted with more 10% methanol/DCM (4×50 ml) and the combined organics dried over Na₂SO₄, filtered and concentrated in vacuo to give a brown gum. This was chromatographed on silica gel eluting with a gradient of 0-20% methanol/DCM to give the title compound as a pale yellow solid. (0.750 g. 20%).

MS (ES+) m/z 415 (MH⁺, 100%).

### (g) 1,1-Dimethylethyl {1-[(3,9-dioxo-6,7-dihydro-3H,5H,9H-pyrimido[1,2,3-ij]-1,8-naphthyridin-5-yl)methyl]-4-piperidinyl}carbamate

1,1-Dimethylethyl {1-[(3,9-dioxo-2,3,6,7-tetrahydro-1H,5H,9H-pyrimido[1,2,3-ij]-1,8-naphthyridin-7-yl)methyl]-4-piperidinyl}carbamate (0.750 g, 1.405 mmol) was dissolved in 1,4-dioxane (20 ml) and treated with 2,3 dichloro,5,6 dicyano-benzoquinone (DDQ) (0.638 g, 2.81 mmol) and heated at 60° C. overnight. The mixture was treated with a 5% solution of potassium carbonate in water (50 ml) and extracted with 20% methanol/DCM (3×50 ml). The combined organics were dried over Na₂SO₄, filtered and concentrated in vacuo. The residue was chromatographed on silica gel eluting with a gradient of 0-20% methanol/DCM to give the title compound as a pale yellow solid. (0.19 g, 30%).

MS (ES+) m/z 415 (MH⁺, 100%).

### (h) 5-[(4-Amino-1-piperidinyl)methyl]-6,7-dihydro-3H,5H,9H-pyrimido[1,2,3-ij]-1,8-naphthyridine-3,9-dione

1,1-Dimethylethyl {1-[(3,9-dioxo-6,7-dihydro-3H,5H,9H-pyrimido[1,2,3-ij]-1,8-naphthyridin-5-yl)methyl]-4-piperidinyl}carbamate (0.19 g, 0.458 mmol) was dissolved in methanol (5 ml) and DCM (5 ml) and treated with 4 M hydrogen chloride in 1,4-dioxane (0.688 ml, 2.75 mmol). The mixture was stirred at RT for 20 mins. The excess solvent was removed in vacuo and the solid dried under high vacuum to give the title compound as a white solid. (0.21 g, 95%).

MS (ES+) m/z 315 (MH⁺, 100%).

### (i) Title Compound

5-[(4-Amino-1-piperidinyl)methyl]-6,7-dihydro-3H,5H,9H-pyrimido[1,2,3-ij]-1,8-naphthyridine-3,9-dione (0.210 g, 0.434 mmol) was dissolved in methanol (16 ml) and acetic acid (0.160 ml) and treated with sodium acetate (0.124 g, 1.518 mmol) and 2,3-dihydro[1,4]oxathiino[2,3-c]pyridine-7-carbaldehyde (for a synthesis see WO2004058144 Example 60) (0.071 g, 0.390 mmol). The mixture was stirred at RT for 20 mins. Sodiumcyanoborohydride (0.027 g, 0.434 mmol) was added and the mixture stirred at RT overnight under argon atmosphere. The mixture was then poured onto a SCX cartridge (2.0 g) and eluted with methanol (10 ml) followed by 0.2M methanolic ammonia (10 ml). Fractions containing the compound were combined together and concentrated in vacuo to give a pale yellow foam. This was chromatographed on silica gel eluting with a gradient of 0-20% MeOH/DCM to give the free base of the title compound as a pale yellow solid. (0.071 g, 32%).

¹HNMR free base (400 MHz, CDCl₃) δ 1.34-1.50 (2H, m), 1.89-1.91 (3H, m), 2.08-2.15 (1H, m), 2.23-2.29 (1H, m), 2.36-2.42 (1H, m), 2.46-2.59 (2H, m), 2.69-2.72 (2H, m), 3.10-3.18 (3H, m), 3.62-3.70 (1H, m), 3.8 (2H, s), 4.40-4.42 (2H, m),4.55-4.60 (2H m), 5.19-5.22 (2H, m), 6.33-6.38 (2H, m), 7.00 (1H, s), 7.43-7.46 (2H, m), 8.02 (1H, s).

MS (ES+), m/z 480 (MH⁺, 70%).

The free base of the title compound was dissolved in methanol and treated with 1 M hydrogen chloride in diethyl ether. The solvent was then evaporated to give the title compound as a yellow solid.

### Example 11

### 5-({4-[([1,3]Oxathiolo[5,4-c]pyridin-6-ylmethyl)amino]-1-piperidinyl}methyl)-6,7-dihydro-3H,5H,9H-pyrimido[1,2,3-ij]-1,8-naphthyridine-3,9-dione dihydrochloride

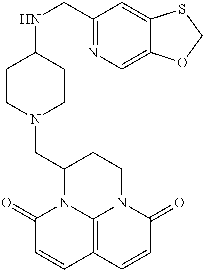

5-[(4-Amino-1-piperidinyl)methyl]-6,7-dihydro-3H,5H,9H-pyrimido[1,2,3-ij]-1,8-naphthyridine-3,9-dione (for a preparation see Example 10(h)) (0.055 g, 0.13 mmol) was dissolved in methanol (16 ml) and acetic acid (0.160 ml) and treated with sodium acetate (0.027 g, 0.33 mmol) and [1,3]oxathiolo[5,4-c]pyridine-6-carbaldehyde (for a synthesis see WO2004058144 Example 61) (0.017 g, 0.1 mmol) and the mixture stirred at RT for 20 mins. Sodium cyanoborohydride (0.008 g, 0.13 mmol) was added and the mixture stirred at RT overnight under argon atmosphere. The mixture was poured on a SCX cartridge (2.0 g) and eluted with methanol (10 ml) followed by 0.2M methanolic ammonia (10 ml) and fractions containing the compound were combined together and concentrated in vacuo to give a pale yellow foam. This was chromatographed on silica gel eluting with a gradient of 0-20% MeOH/DCM to give the free base of the title compound as a pale yellow solid. (0.018 g, 30%).

¹HNMR free base (400 MHz, CDCl₃) δ 1.41-1.48 (2H, m), 1.70 (2H, s), 1.84-1.93 (2H, m), 2.09-2.15 (1H, m), 2.23-2.27 (1H, m), 2.40-2.42 (1H, m), 2.50-2.57 (2H, m), 2.72-278 (2H, m), 3.10-3.14 (1H, m), 3.62-3.67 3 (1H, m),3.82 (2H, s), 4.55-4.59 (1H, m), 5.19-5.22 (1H m), 5.73 (2H, s), 6.33-6.38 (2H, m), 7.30 (1H, s), 7.43-7.46 (2H, m), 8.02 (1H, s).

MS (ES+), m/z 466 (MH⁺, 100%).

The free base of the title compound was dissolved in methanol (2 mL) and treated with excess 1M hydrogen chloride in diethyl ether. The solvent was then evaporated to give the title compound as a yellow solid.

### Example 12

### 5-({4-[(2,3-Dihydro[1,4]dioxino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione dihydrochloride

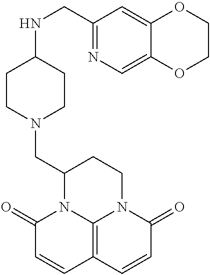

5-[(4-Amino-1-piperidinyl)methyl]-6,7-dihydro-3H,5H,9H-pyrimido[1,2,3-ij]-1,8-naphthyridine-3,9-dione (for a preparation see Example 10(h)) (0.055 g, 0.13 mmol) was dissolved in methanol (4 ml) and acetic acid (0.16 ml) and treated with sodium acetate (0.027 g, 0.33 mmol) and 2,3-dihydro[1,4]dioxino[2,3-c]pyridine-7-carbaldehyde (for a synthesis see WO2003087098 Example 19(d)) (0.016 g, 0.1 mmol) and the mixture stirred for 20 mins. Sodiumcyanoborohydride (0.008 g, 0.13 mmol) was added and the mixture stirred at RT overnight under argon atmosphere. The mixture was poured on a SCX cartridge (2.0 g) and eluted with methanol (10 ml) followed by 0.2M methanolic ammonia (10 ml), fractions containing the compound were combined together and concentrated in vacuo to give a pale yellow foam. This was chromatographed on silica gel eluting with a gradient of 0-20% MeOH/DCM to give the free base of the title compound as a pale yellow solid. (0.017 g, 30%).

¹HNMR (400 MHz, CDCl₃) δ 1.40-1.49 (2H, m), 1.81-1.92 (4H, m), 2.02-214 (1H, m), 2.23-2.29 (1H, m), 2.34-2.40 (1H, m), 2.49-2.50 (2H, m), 2.69-2.70 (2H, m), 3.10-3.13 (1H, m), 3.10-3.14 (1H, m), 3.62-3.67 (1H, m),3.82 (2H, s), 4.22-4.32 (4H, m),4.55-4.61 (1H m), 5.18-5.22 (1H, m), 6.33-6.38 (2H, m), 6.82 (1H, s), 7.43-7.46 (2H, m), 8.10 (1H, s). MS (ES+), m/z 464 (MH⁺, 100%).

The free base of the title compound was dissolved in methanol (2 mL) and treated with excess 1 M hydrogen chloride in diethyl ether. The solvent was then evaporated to give the title compound as a yellow solid.

### Example 13

### 5-({4-[(2,3-Dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione benzoic acid salt enantiomer E1 and

### Example 14

### 5-({4-[(2,3-Dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione benzoic acid salt enantiomer E2

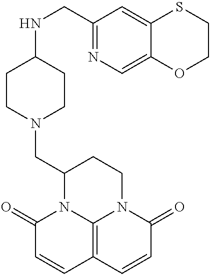

The free base 5-({4-[(2,3-dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione (for a preparation see Example 10) (60 mg) was chromatographed in three runs on Chiralpak AD-H (5 microns, 21 mm×250 mm) eluting at 20 ml/min with 30:30:40:0.1 acetonitrile:methanol:isopropanol:isopropylamine, to separate the enantiomers into E1 (100% ee, elution time 6.9 minutes) and E2 (100% ee, elution time 10.1 minutes). The free base of each final compound in methanol was converted into the monobenzoate salt in methanol. Evaporation followed by trituration with ether and drying in vacuo afforded the title compounds as white solids Enantiomer E1 (29 mg), and Enantiomer E2 (29 mg).

MS (ES+), m/z 480 (MH⁺, 100%).

### Example 15

### 7-{[(1-{[(6S)-3,7-Dioxo-5,6-dihydro-3H,4H,7H-1,3a,6a-triazaphenalen-6-yl]methyl}-4-piperidinyl)amino]methyl}-2,3-dihydro-1,4-benzodioxin-5-carbonitrile dihydrochloride

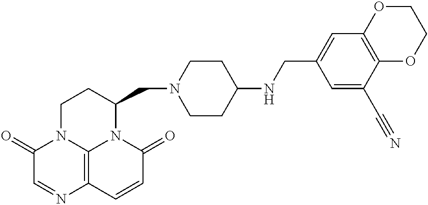

A mixture of (6S)-6-[(4-amino-1-piperidinyl)methyl]-5,6-dihydro-3H,4H,7H-1,3a,6a-triazaphenalene-3,7-dione dihydrochloride (for a preparation see Example 5A(d) (150 mg, 0.386 mmol) and 7-formyl-2,3-dihydro-1,4-benzodioxin-5-carbonitrile (8-cyano-2,3-dihydro-1,4-benzodioxin-6-carbaldehyde, for a synthesis see WO06014580 Preparation 13 or WO2007122258 Example 31(d)) (67 mg, 0.354 mmol) in methanol (15 ml) and acetic acid (75 μl) was treated with sodium acetate (64 mg, 0.780 mmol) and (polystyrylmethy)trimethylammonium cyanoborohydride (580 mg, 2.320 mmol). The resulting mixture was stirred at 20° C. overnight. The mixture was filtered and the beads washed with 1:1 DCM:methanol (20 ml). The combined filtrate and washings were evaporated to dryness. The residue was dissolved in 10:1 DCM:methanol (20 ml) and washed with saturated sodium bicarbonate solution (25 ml) and brine (10 ml) and dried (hydrophobic frit). Solvent was removed to give the crude product. The crude product was purified by column chromatography on silica gel eluting with 0% to 100% of (20% 2M methanolic ammonia in dichloromethane) in dichloromethane to give the free base of the title compound as a pale brown foam (96 mg, 0.197 mmol, 51% yield).

¹H NMR free base (400 MHz, CDCl₃): δ 1.30-1.45 (2H, m), 1.81-2.01 (3H, m), 2.05-2.21 (1H, m), 2.25-2.62 (1H, m), 2.39-2.55 (2H, m), 2.55-2.64 (1H, m), 2.68-2.75 (1H, m), 2.79-2.85 (1H, m), 3.05-3.15 (1H, m), 3.51-3.70 (1H, m), 3.72 (2H, s), 4.25-4.45 (4H, m), 4.50-4.60 (1H, m), 5.11-5.21 (1H, m), 6.46 (1H, d), 7.10 (2H, m), 7.78 (1H, d), 7.95 (1H, s).

MS (ES+), m/z 489 (MH⁺, 100%).

The free base of the title compound was dissolved in methanol (2 ml) and treated with 4M HCl/1,4-dioxane (0.2 ml) then evaporated to dryness to give the title compound as a pale brown solid (79 mg).

### Example 16

### (6S)-6-[(4-{[(7-Fluoro-2,3-dihydro-1,4-benzodioxin-6-yl)methyl]amino}-1-piperidinyl)methyl]-5,6-dihydro-3H,4H,7H-1,3a,6a-triazaphenalene-3,7-dione dihydrochloride

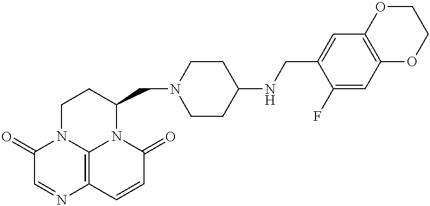

A mixture of 7-fluoro-2,3-dihydro-benzo[1,4]dioxin-6-carboxaldehyde (for a synthesis see WO2002056882, Example 23(a)) (90 mg, 0.494 mmol) and (6S)-6-[(4-amino-1-piperidinyl)methyl]-5,6-dihydro-3H,4H,7H-1,3a,6a-triazaphenalene-3,7-dione, dihydrochloride (for a preparation see Example 5A(d) (220 mg, 0.488 mmol) in a mixture of chloroform (10 ml) and methanol (1 ml) was stirred at 20° C. for 1 h. NaBH(OAc)₃ (282 mg, 1.331 mmol) was added in portions over approximately 5 minutes. The resulting mixture was stirred at 20° C. for 3d. A further quantity of NaBH(OAc)₃ (200 mg) was added and stirring was continued for a further 24 h. Saturated aqueous sodium bicarbonate was added and the mixture was extracted with 20% methanol in dichloromethane (2×25 mL) and the extracts were dried (MgSO₄). The reaction mixture was then evaporated. Purification was carried out by column chromatography on silica gel eluting with 0% to 100% of (20% 2M methanolic ammonia in dichloromethane) in dichloromethane to give the free base of the title compound (122 mg, 0.248 mmol, 50% yield).

¹H NMR free base (400 MHz, CDCl₃): δ 1.30-1.48 (2H, m), 1.81-1.99 (3H, m), 2.07-2.18 (1H, m), 2.21-2.32 (1H, m), 2.32-2.52 (2H, m), 2.52-2.61 (1H, m), 2.62-2.72 (1H, m), 2.75-2.85 (1H, m), 3.04-3.12 (1H, m), 3.52-3.70 (1H, m), 3.72 (2H, s), 4.20-4.29 (4H, m), 4.50-4.58 (1H, m), 5.10-5.19 (1H, m), 6.45 (1H, d), 6.58 (1H, d), 6.81 (1H, d), 7.78 (1H, d), 7.91 (1H, s).

MS (ES+), m/z 482 (MH⁺, 100%).

The free base of the title compound was dissolved in methanol (3 ml) and treated with 4M HCl/1,4-dioxane (0.2 ml) then evaporated to dryness to give the title compound as a pale brown solid (88 mg).

### Example 17A

### (6S)-6-({4-[(2,3-Dihydro-1,4-benzodioxin-6-ylmethyl)amino]-1-piperidinyl}methyl)-5,6-dihydro-3H,4H,7H-1,3a,6a-triazaphenalene-3,7-dione dihydrochloride

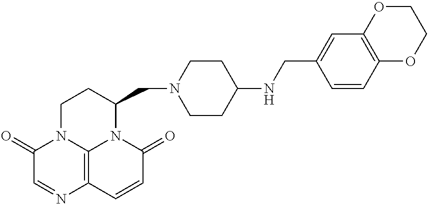

A mixture of 2,3-dihydro-1,4-benzodioxin-6-carbaldehyde (commercially available) (90 mg, 0.548 mmol) and (6S)-6-[(4-amino-1-piperidinyl)methyl]-5,6-dihydro-3H,4H,7H-1,3a,6a-triazaphenalene-3,7-dione dihydrochloride (for a preparation see Example 5A(d) (220 mg, 0.488 mmol) in methanol (1 ml) was stirred at 20° C. NaBH(OAc)₃ (282 mg, 1.331 mmol) was added in portions over approximately 5 minutes. The resulting mixture was stirred at 20° C. for 3d. A further quantity of NaBH(OAc)₃ (200 mg) was added and stirring was continued for a further 24 h. Saturated aqueous sodium bicarbonate was added and the mixture was extracted with 20% methanol in dichloromethane (2×25 mL) and the extracts were dried (MgSO₄). The reaction mixture was then evaporated. Purification was carried out by column chromatography on silica gel eluting with 0% to 100% of (20% 2M methanolic ammonia in dichloromethane) in dichloromethane to give the free base of the title compound (122 mg, 0.248 mmol, 50% yield).

¹H NMR free base (400 MHz, CDCl₃): δ 1.30-1.50 (2H, m), 1.81-2.00 (3H, m), 2.00-2.19 (1H, m), 2.22-2.35 (1H, m), 2.36-2.45 (1H, m), 2.45-2.62 (2H, m), 2.65-2.71 (1H, m), 2.75-2.85 (1H, m), 3.04-3.15 (1H, m), 3.55-3.70 (1H, m), 3.70 (2H, s), 4.26 (4H, m), 4.50-4.60 (1H, m), 5.11-5.21 (1H, m), 6.45 (1H, d), 6.72-6.85 (3H, m), 7.78 (1H, d), 7.92 (1H, s).

MS (ES+), m/z 464 (MH⁺, 100%).

The free base was dissolved in methanol (3 mL) and treated with 4M hydrogen chloride in 1,4-dioxane (0.2 mL) to give the title compound (87.5 mg, 0.160 mmol, 32.7% yield) as a pale brown solid.

### Example 17B

### (6S)-6-({4-[(2,3-Dihydro-1,4-benzodioxin-6-ylmethyl)amino]-1-piperidinyl}methyl)-5,6-dihydro-3H,4H,7H-1,3a,6a-triazaphenalene-3,7-dione hydrochloride

The free base of Example 17A was dissolved in DCM/MeOH and treated with 1 eq of 1M HCl in Et₂O. The solvents were evaporated and the solid was dried in the desiccator (P₂O₅) overnight to afford the title hydrochloride salt as an off-white solid.

MS (ES+), m/z 464 (MH⁺, 100%).

### Preparation A 6-(Methyloxy)pyrido[2,3-b]pyrazin-3(4H)-one

### (a) 6-(Methyloxy)-3-nitro-2-pyridinamine

A solution/suspension of 2-chloro-6-(methyloxy)-3-nitropyridine (65.7 g, 348 mmol) in 2M ammonia in methanol (500 ml, 1000 mmol) and aqueous ammonia (500 ml, 348 mmol) was stirred at 65° C. for 18 h. The reaction was cooled down and the solid filtered off and washed with water (2×100 ml). The solid was dried in the vacuum oven at 40° C. overnight to afford the product as a bright yellow solid (52.14 g, 84% purity by NMR, 74%).

MS (ES+) m/z 170 (MH⁺).

### (b) 6-(Methyloxy)-2,3-pyridinediamine

6-(Methyloxy)-3-nitro-2-pyridinamine (26 g, 129 mmol) was suspended in ethanol (500 ml) at room temperature under argon and then treated with palladium on carbon (15 g, 14.10 mmol) (10% paste). The reaction was stirred under 1 atm of hydrogen overnight. The reaction was filtered through a Celite pad and the pad washed with ethanol (500 ml). Ethanol was evaporated to afford the product as a purple oil (20.68 g, slightly impure).

MS (ES+) m/z 140 (MH⁺).

### (c) Ethyl N-[2-amino-6-(methyloxy)-3-pyridinyl]glycinate

6-(Methyloxy)-2,3-pyridinediamine (21.7 g, estimated 87% purity, 136 mmol) was dissolved in acetonitrile (500 ml) at room temperature under argon and then treated with potassium carbonate (24.38 g, 176 mmol) and ethyl bromoacetate (18.13 ml, 163 mmol). The reaction was stirred at room temperature overnight. The acetonitrile was then removed in vacuo. The reaction was repeated using more 6-(methyloxy)-2,3-pyridinediamine (20.68 g, 87% purity, 129 mmol), in acetonitrile (500 ml), potassium carbonate (23.23 g) and ethyl bromoacetate (17.27 g) and the reaction was again stirred at room temperature overnight and the acetonitrile was then removed in vacuo. The residues were partitioned between water (1 L) and ethyl acetate (1 L) and the layers separated. The aqueous layer was extracted once more with ethyl acetate (1 L) and the combined organic extracts were dried over MgSO₄, filtered and evaporated to afford a purple oil (64 g). The oil was treated with DCM (300 ml) and the insoluble impurities filtered off. The DCM solution was loaded onto a 800 g silica column and eluted with 0-2% MeOH/DCM to afford 40.6 g of desired product as a brown solid (LCMS and NMR consistent with 75% desired product with 15% cyclized product 6-(methyloxy)-1,4-dihydropyrido[2,3-b]pyrazin-3(2H)-one and 6.4 g of cyclized product 6-(methyloxy)-1,4-dihydropyrido[2,3-b]pyrazin-3(2H)-one as a purple solid.

MS (ES+) m/z 226 (MH⁺).

### (d) 6-(Methyloxy)-1,4-dihydropyrido[2,3-b]pyrazin-3(2H)-one

Ethyl N-[2-amino-6-(methyloxy)-3-pyridinyl]glycinate (40.6 g, 135 mmol) was dissolved in tetrahydrofuran (THF) (1 L) at room temperature under argon and treated with potassium tert-butoxide (15.17 g, 135 mmol). After 2 h at room temperature saturated NH₄Cl (500 ml) was added and the THF evaporated. Water (500 ml) was added followed by 20% MeOH/DCM (1 L); the insoluble material was filtered off, washed with diethyl ether and dried in the vacuum oven at 40° C. overnight to afford the desired product as a yellow solid (15.3 g): LCMS and NMR consistent with product (9% of oxidized material present by NMR).

The two phases were transferred to a separating funnel and separated. The aqueous layer was extracted twice more with 20% MeOH/DCM (2×500 ml) and the combined organic extracts were dried, MgSO₄ filtered and evaporated to afford a brown solid which was washed with plenty of diethyl ether to afford more of the desired product as a pale green solid (7.7 g): LCMS and NMR consistent with product (20% of oxidized material present by NMR).

MS (ES+) m/z 180 (MH⁺).

### Alternative Procedure:

Ethyl N-[2-amino-6-(methyloxy)-3-pyridinyl]glycinate (16.2 g, 72 mmol) was dissolved in tetrahydrofuran (500 ml) and cooled to 0° C. (ice bath cooling) under argon. This was then treated with potassium tert-butoxide (1M in THF, 80 ml, 80 mmol). After 1.5 h the reaction was treated with acetic acid (80 mmol) and evaporated to give a dark solid. This was triturated with water (200 ml), filtered and dried in vacuo (˜13 g, quant.), which may be used without further purification

### (e) 6-(Methyloxy)pyrido[2,3-b]pyrazin-3(4H)-one

A solution of 6-(methyloxy)-1,4-dihydropyrido[2,3-b]pyrazin-3(2H)-one (10.9 g, 61 mmol) in DMF (200 ml) was treated with MnO₂ (26.4 g). After 2 hours at room temperature no reaction had occurred so more MnO₂ (18 g) was added and the mixture heated at 50° C. for 3 hours. The reaction mixture was filtered through Celite, washing with warm DMF (2×100 ml). The collected solution was concentrated to ca 100 ml, whereupon precipitation of the product commenced. Diluting slowly with water (200 ml) and then filtering the precipitate which was dried in vacuo gave the product as a light brown solid (7.3 g, 68%).

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/ml.

Compounds were evaluated against a panel of Gram-positive organisms, selected from Staphylococcus aureus, Streptococcus pneumoniae, Streptococcus pyogenes, Enterococcus faecalis and Enterococcus faecium.

In addition, compounds were evaluated against Gram-negative organisms selected from Haemophilus influenzae, Moraxella catarrhalis, Escherichia coli, Pseudomonas aeruginosa, Proteus mirabilis, Enterobacter cloacae, Enterobacter aerogenes, Klebsiella pneumoniae, Stenotrophomonas maltophilia and Acinetobacter baumanii.

The minimum inhibitory concentration (MIC) was determined as the lowest concentration of compound that inhibited visible growth. A mirror reader was used to assist in determining the MIC endpoint.

Each of the listed Examples, as identified in the present application, was tested in at least one exemplified salt or free base form. Unless otherwise noted, the tested Examples had a MIC≦1 μg/ml against a strain of at least one of the organisms listed above. For at least one strain of every organism listed above, at least one Example had a MIC≦2 μg/ml.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Examples 1, 2, 4-9, 15, 16 and 17B were tested in the Mycobacterium tuberculosis H37Rv inhibition assay. Examples 1, 2, 8, 9 and 16 showed an MIC value higher than 4 ug/ml. Examples 5-7, 11, 15 and 17B showed an MIC value lower than 4 ug/ml. Example 4 showed an MIC value lower than 0.4 mg/ml.

1. A compound of formula (I) or a pharmaceutically acceptable salt and/or N-oxide thereof: 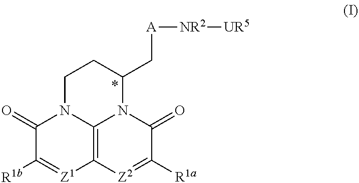
wherein:
Z¹ and Z² are selected from CH and N;
one of R^{1a} and R^{1b} is selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted (C₁₋₆)alkyl; (C₁₋₆)alkoxy-substituted (C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl; and the other is hydrogen;
provided that R^{1a} and R^{1b} are H when Z² or Z¹ is N, respectively, and R^{1b} is H when Z² and Z¹ are both CH;
R² is hydrogen, or (C₁₋₄)alkyl, or together with R⁶ forms Y as defined below;
A is a group (i): 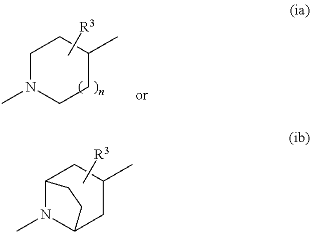
in which: R³ is as defined for R^{1a} and R^{1b} or is oxo and n is 1 or 2:
or A is a group (ii) 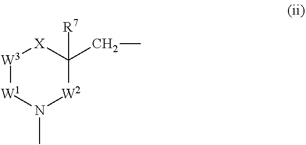
W¹, W² and W³ are CR⁴R⁸
or W² and W³ are CR⁴R⁸ and W¹ represents a bond between W³ and N.
X is O, CR⁴R⁸, or NR⁶;
one R⁴ is as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
U is selected from CO, and CH₂ and
R⁵ is an optionally substituted bicyclic carbocyclic or heterocyclic ring system (B): 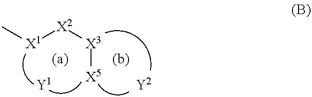
containing up to four heteroatoms in each ring in which
at least one of rings (a) and (b) is aromatic;
X¹ is C or N when part of an aromatic ring, or CR¹⁴ when part of a non-aromatic ring;
X² is N, NR¹³, O, S(O)ₓ, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring;
Y² is a 2 to 6 atom linker group, each atom of Y² being independently selected from N, NR¹³, O, S(O)ₓ, CO, CR¹⁴ when part of an aromatic or non-aromatic ring or may additionally be CR¹⁴R¹⁵ when part of a non aromatic ring;
each of R¹⁴ and R¹⁵ is independently selected from: H; (C₁₋₄)alkylthio; halo; carboxy(C₁₋₄)alkyl; (C₁₋₄)alkyl; (C₁₋₄)alkoxycarbonyl; (C₁₋₄)alkylcarbonyl; (C₁₋₄)alkoxy (C₁₋₄)alkyl; hydroxy; hydroxy(C₁₋₄)alkyl; (C₁₋₄)alkoxy; nitro; cyano; carboxy; amino or aminocarbonyl optionally mono- or di-substituted by (C₁₋₄)alkyl; or
R¹⁴ and R¹⁵ may together represent oxo;
each R¹³ is independently H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; (C₁₋₆)alkoxycarbonyl; (C₁₋₄)alkylcarbonyl; (C₁₋₆)alkylsulphonyl; aminocarbonyl wherein the amino group is optionally mono or disubstituted by (C₁₋₄)alkyl; and
each x is independently 0, 1 or 2.

2. A compound according to claim 1 wherein:
(i) Z¹ and Z² are both CH;
(ii) Z¹ is N and Z² is CH; or
(iii) Z¹ is CH and Z² is N.
and R^{1a} and R^{1b} are hydrogen.

3. A compound according to claim 1 wherein A is a group (ia) in which n is 1 and R³ is hydrogen or hydroxy.

4. A compound according to claim 1 wherein A is (ii), X is CR⁴R⁸, R⁸ is H and R⁴ is H or OH, W¹ is a bond, W² and W³ are both CH₂ and R⁷ is H, or X is O, R⁷ is H and W¹, W² and W³ are each CH₂.

5. A compound according to claim 1 wherein R² is hydrogen.

6. A compound according to claim 1 wherein U is CH₂.

7. A compound according to claim 1 wherein R⁵ is an aromatic heterocyclic ring (B) having 8-11 ring atoms including 2-4 heteroatoms of which at least one is N or NR¹³ in which Y² contains 2-3 heteroatoms, one of which is S and 1-2 are N, with one N bonded to X³, or the heterocyclic ring (B) has ring (a) aromatic selected from optionally substituted benzo, pyrido, pyridazino and pyrimidino and ring (b) non aromatic and Y² has 3-5 atoms, including at least one heteroatom, with O, S, CH₂ or NR¹³ bonded to X⁵ where R¹³ is other than hydrogen, and either NHCO bonded via N to X³, or O, S, CH₂ or NH bonded to X³.

8. A compound according to claim 1 wherein R⁵ is selected from:
3-oxo-3,4-dihydro-2H-pyrido[3,2-b][1,4]oxazin-6-yl
3-oxo-3,4-dihydro-2H-pyrido[3,2-b][1,4]thiazin-6-yl
2,3-dihydro-[1,4]dioxino[2,3-c]pyridin-7-yl
[1,3]oxathiolo[5,4-c]pyridin-6-yl
6-fluoro-2,3-dihydro-1,4-benzodioxin-7-yl
2,3-dihydro[1,4]oxathiino[2,3-c]pyridin-7-yl
3,4-dihydro-2H-pyrano[2,3-c]pyridin-6-yl

9. A compound according to claim 1 selected from:
6-({4-[(2,3-dihydro[1,4]dioxino[2,3-c]pyridin-7-ylmethyl)amino]-1-piperidinyl}methyl)-5,6-dihydro-3H,4H,7H-1,3a,6a-triazaphenalene-3,7-dione;
6-({4-[([1,3]oxathiolo[5,4-c]pyridin-6-ylmethyl)amino]-1-piperidinyl}methyl)-5,6-dihydro-3H,4H,7H-1,3a,6a-triazaphenalene-3,7-dione;
6-({4-[(2,3-dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione;
6-[(4-{[(3-oxo-3,4-dihydro-2H-pyrido[3,2-b][1,4]thiazin-6-yl)methyl]amino}-1-piperidinyl)methyl]-5,6-dihydro-3H,4H,7H-1,3a,6a-triazaphenalene-3,7-dione;
(6S)-6-({4-[(2,3-dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione;
(6S)-6-({4-[(3,4-dihydro-2H-pyrano[2,3-c]pyridin-6-ylmethyl)amino]-1-piperidinyl}methyl)-5,6-dihydro-3H,4H,7H-1,3a,6a-triazaphenalene-3,7-dione;
(6S)-6-({4-[([1,3]oxathiolo[5,4-c]pyridin-6-ylmethyl)amino]-1-piperidinyl}methyl)-5,6-dihydro-3H,4H,7H-1,3a,6a-triazaphenalene-3,7-dione;
4-({4-[([1,3]oxathiolo[5,4-c]pyridin-6-ylmethyl)amino]-1-piperidinyl}methyl)-5,6-dihydro-3H,4H,7H-1,3a,6a-triazaphenalene-3,7-dione;
(4S)-4-({4-[(2,3-dihydro[1,4]oxathiino[2,3-c]pyridin-7-ylmethyl)amino]-1-piperidinyl}methyl)-5,6-dihydro-3H,4H,7H-1,3a,6a-triazaphenalene-3,7-dione;
5-({4-[(2,3-dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione;
5-({4-[([1,3]oxathiolo[5,4-c]pyridin-6-ylmethyl)amino]-1-piperidinyl}methyl)-6,7-dihydro-3H,5H,9H-pyrimido[1,2,3-ij]-1,8-naphthyridine-3,9-dione;
5-({4-[(2,3-dihydro[1,4]dioxino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione;
5-({4-[(2,3-dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione enantiomer E1;
5-({4-[(2,3-dihydro[1,4]oxathiino[2,3-c]pyridin-7-ylmethyl)amino]-1-piperidinyl}methyl)-6,7-dihydro-3H,5H,9H-pyrimido[1,2,3-ij]-1,8-naphthyridine-3,9-dione enantiomer E2;
7-{[(1-{[(6S)-3,7-dioxo-5,6-dihydro-3H,4H,7H-1,3a,6a-triazaphenalen-6-yl]methyl}-4-piperidinyl)amino]methyl}-2,3-dihydro-1,4-benzodioxin-5-carbonitrile;
(6S)-6-[(4-{[(7-fluoro-2,3-dihydro-1,4-benzodioxin-6-yl)methyl]amino}-1-piperidinyl)methyl]-5,6-dihydro-3H,4H,7H-1,3a,6a-triazaphenalene-3,7-dione; or
(6S)-6-({4-[(2,3-dihydro-1,4-benzodioxin-6-ylmethyl)amino]-1-piperidinyl}methyl)-5,6-dihydro-3H,4H,7H-1,3a, 6a-triazaphenalene-3,7-dione;
or a pharmaceutically acceptable salt thereof.

10. A method of treatment of bacterial infections in mammals, particularly in man, which method comprises the administration to a mammal in need of such treatment an effective amount of a compound according to claim 1.

11-13. (canceled)

14. A pharmaceutical composition comprising a compound according to claim 1 and a pharmaceutically acceptable carrier.

15. A compound of formula (IV): 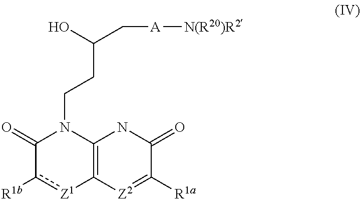
in which R²⁰ is hydrogen, R^{2′} is R² or a group convertible thereto and the remaining variables are as defined in claim 1.

16. A compound of formula (V): 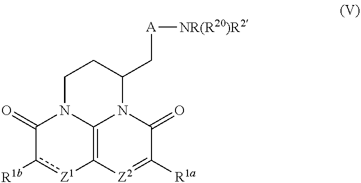
in which R²⁰ is hydrogen, R^{2′} is R² or a group convertible thereto and the remaining variables are as defined in claim 1.
